(19) United States
(12) Patent Application Publication
(10) Pub. No.: US 2010/0183585 A1
(43) Pub. Date: Jul. 22, 2010

(54) METHODS AND COMPOSITIONS FOR TREATING TUMORS AND METASTASES THROUGH THE MODULATION OF LATEXIN

(75) Inventors: Gary Van Zant, Lexington, KY (US); Ying Liang, Lexington, KY (US)

Correspondence Address: CROWELL & MORING LLP;INTELLECTUAL PROPERTY GROUP, P.O. BOX 14300, WASHINGTON, DC, 20044-4300, US

(73) Assignee: University of Kentucky Research Foundation, Lexington, KY (US)

(21) Appl. No.: 12/608,376

(22) Filed: Oct. 29, 2009

Related U.S. Application Data
(60) Provisional application No. 61/109,707, filed on Oct. 30, 2008.

(51) Int. Cl. A61K 39/395; A61P 35/00; A61P 35/04; A61P 35/02

(52) U.S. Cl. 424/130.1

(57) ABSTRACT

The present invention relates to methods for treating cancers and metastatic diseases by modulating latexin expression and/or latexin activity.

### CROSS-REFERENCE TO RELATED APPLICATIONS

This application claims priority under 35 U.S.C. §119 to U.S. Provisional Application No. 61/109,707, filed on Oct. 30, 2008, the entire content of which is hereby incorporated by reference in its entirety.

### STATEMENT OF GOVERNMENT INTEREST

This invention has been made with Government support under grant number 1 RO1 AG02490, awarded by the National Institutes of Health of the United States. The government has certain rights in the invention.

### TECHNICAL FIELD

The present invention relates to methods for treating cancers and metastatic diseases by modulating latexin expression and/or latexin activity.

### BACKGROUND OF THE INVENTION

Many tumors originate from the malignant transformation of resident adult stem and progenitor cells (Dick, J. E., et al., (2005) Int J Hematol 82, 389-396; Dick, J. E., et al., (2005) Int J Hematol 82, 389-396; Marx, J. (2007) Science 317, 1029-1031; Bonnet, D. et al., (1997) Nature Med. Vol 3, Iss 7, 730-737; and Clarke, M. F., et al. (2006) Cell 124, 1111-1115). This paradigm is well established in leukemia and some types of solid tumors. Unrestrained stem cell expansion carries with it the risk of mutations, genomic instability, and carcinogenesis.

Recently, it was found that latexin naturally regulates of the size of the hematopoietic stem cell population in mice by influencing self-renewal and apoptosis. The structural similarity and close genetic linkage of latexin to the tumor suppressor, tazarotene-induced gene 1 (TIG1), has led to the discovery that latexin has tumor suppressor properties.

Thus, agents, compositions and methods for inhibiting cancer growth and metastasis, and for the treatment of cancer, through the modulation of latexin are needed, which can be used alone or in combination with other agents.

### SUMMARY OF THE INVENTION

The invention provides for new methods, compositions, and combination therapies for treating tumors and/or metastatic disease and/or inhibiting growth of tumors. The methods, compositions and combination therapies are preferably directed towards the treatment of such cancers as lymphomas, leukemias, melanomas, and metastatic disease of any primary tumor.

In one embodiment, a method of treating cancer is provided, comprising administering a pharmaceutical composition comprising an agent which increases the expression and/or the activity of latexin, wherein latexin includes a latexin polynucleotide variant and/or a latexin polypeptide variant that interacts with the agent or substitutes and derivatives thereof. The latexin polynucleotide variant may have at least 70% sequence similarity to SEQ ID NO: 1. The latexin polypeptide variant may have at least 70% sequence similarity to SEQ ID NO: 2. The cancer may be hematopoietic. The hematopeotic cancer is lymphoma or leukemia.

In another embodiment, a method of inhibiting tumor growth and/or metastases is provided, comprising administering an agent which increases the expression and/or the activity of latexin to a subject in need thereof in a therapeutically effective amount sufficient to inhibit tumor growth and/or metastases, wherein latexin includes a latexin polynucleotide variant and/or a latexin polypeptide variant that interacts with the agent.

In another embodiment, a method of promoting apoptosis in tumor cells is provided, comprising administering an agent which increases the expression and/or the activity of latexin to a subject in need thereof in a therapeutically effective amount to promote apoptosis in tumor cells, wherein latexin includes a latexin polynucleotide variant and/or a latexin polypeptide variant that interacts with the agent. The leukemia may be chronic myelogenous leukemia, acute myelogenous leukemia, adult acute lymphoblastic leukemia, mature B-cell acute lymphoblastic leukemia, chronic lymphocytic leukemia, prolymphocytic leukemia, or hairy cell leukemia. The tumor may be a melanoma and the subject is further subjected to surgery, isolated limb perfusion, regional chemotherapy infusion, systemic chemotherapy, or immunotherapy with a second antibody or antisera to treat the melanoma. The metastases may be a metastasis to brain, lung, liver, or bone. The metastasis may be to lung, and the tumor may be a melanoma.

In another embodiment, a combination therapy for inhibiting tumor growth and/or metastatic progression and/or development of metastases is provided, comprising administering an agent which increases the expression and/or the activity of latexin, wherein latexin includes a latexin polynucleotide variant and/or a latexin polypeptide variant that interacts with the agent; and a chemotherapeutic, an immunotherapeutic, and/or radiation therapy.

### BRIEF DESCRIPTION OF THE DRAWINGS

FIG. 1 shows a decrease or loss of latexin expression in leukemic and lymphoma cells. FIG. 1(a) shows latexin mRNA expression in leukemia cell lines, bone marrow, and peripheral blood CD34+ cells from leukemia and lymphoma patients and normal individuals. Latexin mRNA level was measured by quantitative real-time PCR, and is shown as a mean (±1 SD) (n=12). The patient samples included acute myeloid leukemia (AML), T cell pro-lympho leukemia (Pctl), plasma cell leukemia (PCL), acute T cell lymphoma (ATLL), and acute lymphoid leukemia (ALL, preB phenotype). FIG. 1(b) shows latexin protein expression in leukemic cell lines measured by western blot. The blots (bottom) and their quantification (top) profiles demonstrate the absent or weak expression of latexin protein in leukemic and lymphoma cell lines. Actin was used as the internal control. FIG. 1(c) shows latexin protein expression in bone marrow and peripheral blood CD34+ cells from leukemia and lymphoma patients and normal individuals. A western blot was performed on the corresponding samples shown in panel (a) plus five more samples. The blots (bottom) and their quantification (top) profiles demonstrate the significantly decreased latexin protein level in leukemic CD34+ and lymphoma cells (P=0.03).

FIG. 2 shows the methylation of the latexin promoter CpG sites in leukemic cell lines. FIG. 2(a) shows CpG-enriched region in the latexin promoter. Sequence from the 1500 base pairs upstream regulatory region to the first three exons was analyzed for CpG-enriched sites (top). A CpG island with 252 base pairs (bpₛ) length was identified. The transcription starting site is indicated. Each filled circle represents a CpG dinucleotide (15 total). FIG. 2(b) shows bisulfite sequencing for latexin promoter CpG island methylation analysis in leukemic cell lines. Files are each of the 15 CpGs, and ranks are replicate clones sequenced for each CpG (n=3); open and filled circles indicate unmethylated and methylated CpG dinucleotides, respectively. The methylation content was quantified by dividing the number of methylated CpGs by the total number of analyzed CpGs. FIG. 2(c) shows the restoration or up-regulation of Lxn expression by 5-aza-2′-deoxycytidine. latexin mRNA was measured by quantitative real-time PCR in leukemic and lymphoma cell lines treated with or without 2 μM 5-aza-2′-deoxycytidine for 4 days. Expression levels were normalized using the endogenous control, GAPDH. Results shown are mean±SD of 12 replicates from 3 independent experiments.

FIG. 3 shows methylation of site-specific CpGs in latexin promoter in normal and leukemic cells. Bisulfite sequencing PCR was performed on early progenitor cells of normal young (Y1) and old (O1) bone marrows, as well as CD34+CD38− leukemic stem cells of two AML (1 and 2) patients. Ranks of circles represent each of the 15 CpGs. Files are replicate clones sequenced for each CpG (n=8-9). Open circles and filled circles represent unmethylated and methylated CpG dinucleotides, respectively.

FIG. 4 shows an experimental scheme for the effects of Lxn over-expression on the growth of A20 tumor cell line. AZO cells were retrovirally infected in vitro with control or Lxn-containing vectors. GFP positive cells were purified by sorting, and their growth patterns in vitro and in vivo were determined.

FIG. 5 shows Lxn over-expression suppresses growth of A20 lymphoma cell lines. FIG. 5(a) shows in vitro growth inhibitory effects of Lxn over-expression on A20 cells. A20 cells, uninfected or infected with empty or Lxn-containing vector, were cultured for 20 days and counted by the trypan blue exclusion method. At the indicated time-points (X axis), the absolute cell numbers are presented on a log scale (Y axis; left panel). The tumor growth of Lxn-over-expressing A20 cells relative to the controls is shown in the right panel. FIG. 5(b) shows the proportion of GFP+ A20 cells infected with empty (light gray column) or Lxn-containing vector (black column) throughout 20 days of culture. Flow cytometric analysis was performed to detect the GFP signal. FIG. 5(c) shows western blot analysis using latexin antibody and total protein lysate from A20 cells infected with empty (vector control) or Lxn-containing vector at the 20^{th} day of culture reveals durable Lxn over-expression. FIG. 5(d) shows in vivo tumor formation and growth with injection of 100,000 A20 cells uninfected (control), or infected with either empty (vector control) or Lxn-containing vector. A20 cells were subcutaneously injected, and tumor growth was monitored and measured (all three dimensions; Y axis) on the indicated days (X axis). Shown is the compilation of 2 independent experiments with ±SD (n=12). FIG. 5(e) shows in vivo tumor formation and growth of graded doses of A20 cells. Graded doses of A20 cells (X axis) were subcutaneously injected and tumor growth was monitored and measured (all three tumor dimensions; Y axis) on the indicated days (Z axis). Unfilled columns represent A20 cells infected with empty vector, and filled columns represent cells with Lxn-containing vector. FIG. 5(f) shows the fraction of GFP-expressing A20 cells in tumors biopsied at 21 days. On day 21 (post-injection), single tumor cell suspensions were prepared from mice injected with either uninfected A20 cells (A20 control) or with infected A20 cells harboring empty vector (vector control) or Lxn-containing vector (Lxn vector). This suspension was subjected to flow cytometric analysis for GFP-expressing cells.

FIG. 6 shows potential mechanisms involved in the inhibition of Lxn in A20 lymphoma growth. FIG. 6(a) shows flow cytometric analysis of cell cycle and apoptosis in cultured A20 cells. Uninfected A20 cells (A20 control) and infected A20 cells harboring either empty vector (vector control) or Lxn-containing vector (Lxn vector) were cultured for 21 days. BrdU and 7AAD staining and flow cytometric analysis were used for detecting the different phases of the cell cycle (G0/G1, S, G2/M) and the apoptotic (A) and necrotic (N) populations according to BrdU incorporation and total DNA content (7AAD staining). A representative flow cytometric file from Day 5 of culture is shown, along with the proportion of apoptotic cells in each treatment. FIG. 6(b) shows proportion of apoptotic A20 cells throughout 21 days of culture. The time-points with more than a 2-fold difference between controls and Lxn-over-expression cells are indicated with*. FIG. 6(c) shows the growth curve of A20 cells treated with graded doses of potato carboxypeptidase inhibitor (PCPI).

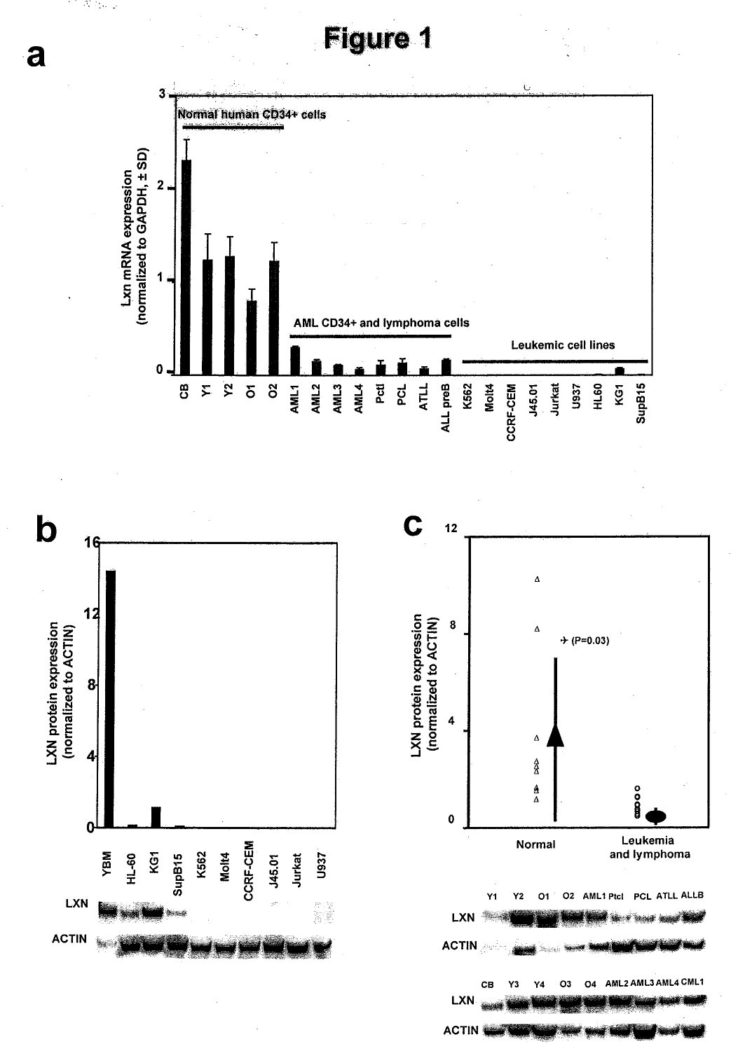

### DETAILED DESCRIPTION OF THE INVENTION

### Definitions

In accordance with this detailed description, the following abbreviations and definitions apply. It must be noted that as used herein, the singular forms “a”, “and”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

By the term “subject” or “patient” as used herein is meant to include a mammal. The mammal can be a canine, feline, primate, bovine, ovine, porcine, camelid, caprine, rodent, or equine. Preferably the mammal is human.

The term “efficacy” as used herein refers to the effectiveness of a particular treatment regime. Efficacy can be measured based on such characteristics (but not limited to these) as inhibition of tumor growth, reduction of tumor mass, reduction of metastatic lesions as assessed by radiologic imaging, slowed tumor growth, lack of detectable tumor associated antigens, and the like. Additional methods of assessing tumor progression are discussed herein and would be known to the treating and diagnosing physicians.

By the phrases “pharmaceutically acceptable carrier” and “pharmaceutically acceptable excipient” are intended to mean any compound(s) used in forming a part of the formulation that is intended to act merely as a carrier, i.e., not intended to have biological activity itself. The pharmaceutically acceptable carrier or excipient is generally safe, non-toxic, and neither biologically nor otherwise undesirable. A pharmaceutically acceptable carrier or excipient as used herein includes both one and more than one such carrier or excipient.

The term “latexin” (“Lxn”) refers to the Lxn gene, isoforms/variants of the Lxn gene, and gene products derived from the Lxn gene, including messenger RNA and protein. The sense-strand of a human latexin cDNA is provided as SEQ ID NO:1. A human latexin protein sequence is provided as SEQ ID NO:2. Latexin isoforms/variants include genes containing exon sequences having at least about 70%, 75%, 80%, 85%, 90%, 95%, 96%, 97%, 98%, or 99% sequence similarity to SEQ ID NO:1; genes containing exon sequences having at least about 70%, 75%, 80%, 85%, 90%, 95%, 96%, 97%, 98%, or 99% sequence identity to SEQ ID NO:1; those sequences encoded by genes containing exon sequences having at least about 70%, 75%, 80%, 85%, 90%, 95%, 96%, 97%, 98%, or 99% sequence similarity to SEQ ID NO:1; those sequences encoded by genes containing exon sequences having at least about 70%, 75%, 80%, 85%, 90%, 95%, 96%, 97%, 98%, or 99% sequence identity to SEQ ID NO:1; those sequences having at least about 70%, 75%, 80%, 85%, 90%, 95%, 96%, 97%, 98%, or 99% sequence similarity to SEQ ID NO:2; and those sequences having at least about 70%, 75%, 80%, 85%, 90%, 95%, 96%, 97%, 98%, or 99% sequence identity to SEQ ID NO:2. Latexin isoforms/variants include mammalian homologs listed in Tables 1-3 as described in [0076]. Examples of latexin isoforms/variants include the carboxypeptidase inhibitor (CARIN) described in U.S. Pat. No. 5,998,373.

Homology can be determined by various methods, including alignments of open-reading-frames (“ORFs”) contained in private and/or public databases. Any suitable mathematical algorithm may be used to determine percent identities and percent similarities between any two sequences being compared. For example, nucleic acid and protein sequences of the present invention can be used as a “query sequence” to perform a search against sequences deposited within various public databases to identify other family members or evolutionarily-related sequences. Genomic sequences for various organisms are currently available, including fungi, such as the budding yeast, or Saccharomyces cerevisiae; invertebrates, such as Caenorhabditis elegans and Drosophila melangaster; and mammals, such as the mouse, rat, and human. Exemplary databases for identifying orthologs of interest include Genebank, Swiss Protein, EMBL, and National Center for Biotechnology Information (“NCBI”), and many others known in the art. These databases enable a user to set various parameters for a hypothetical search according to the user's preference, or to utilize default settings. Tables 1-3, provided below, lists the accession numbers and gene identification numbers for exemplary mammalian orthologs.

TABLE 1
Gene Bank Accession Number for Exemplary Mouse, Human,
and Rat Latexin mRNA.

| Organism | Accession Number | Gene IDS |
|---|---|---|
| Mus musulus | NM-016753 | 31980631 |
| (Mouse) | | |
| Rattus | NM-031655 | 14269567 |
| norvegicus (Rat) | | |
| Homo Sapiens | NM-020169 | 21359932 |
| (human) | | |

TABLE 2
Gene Bank Accession Number for Exemplary Mouse, Human,
and Rat Latexin Protein.

| Organism | Accession Number | Gene IDS |
|---|---|---|
| Mus musulus | NP-058033 | 31980632 |
| (Mouse) | | |
| Rattus | NP-113843 | 14269568 |
| norvegicus (Rat) | | |
| Homo Sapiens | NP-064554 | 21359933 |
| (human) | | |

TABLE 3
Mammalian Homologs for Latexin

| | Accession | | Percentage | |
|---|---|---|---|---|
| Organism | Number | Gene IDS | Identity | Gene Information |
| Mus musulus | AK032170.1 | 26327996 | 99.91 | Mus musculus adult male olfactory |
| (Mouse) | | | | brain cDNA, RIKEN full-length |
| | | | | enriched library, clone: 6430407E02 |
| | | | | product: Latexin, full insert sequence |
| Mus musulus | AK149981.1 | 74211713 | 100.00 | Mus musculus bone marrow macrophage |
| (Mouse) | | | | cDNA, RIKEN full-length enriched |
| | | | | library, clone: G530111O19 product: |
| | | | | Latexin, full insert sequence |
| Mus musulus | D88769.1 | 1669620 | 99.50 | Mus musculus mRNA for Latexin, |
| (Mouse) | | | | complete cds |
| Mus musulus | AC124190.4 | 23499687 | 100.00 | Mus musculus BAC clone RP23-267M9 |
| (Mouse) | | | | from 3, complete sequence |
| Mus musulus | AC124190.4 | 23499687 | 100.00 | |
| (Mouse) | | | | |
| Mus musulus | AC124190.4 | 23499687 | 100.00 | |
| (Mouse) | | | | |
| Mus musulus | AC124190.4 | 23499687 | 100.00 | |
| (Mouse) | | | | |
| Mus musulus | AC124190.4 | 23499687 | 100.00 | |
| (Mouse) | | | | |
| Mus musulus | AC124190.4 | 23499687 | 100.00 | |
| (Mouse) | | | | |
| Mus musulus | AK018305.1 | 12857946 | 99.58 | Mus musculus 10 days neonate |
| (Mouse) | | | | cerebellum cDNA, RIKEN full-length |
| | | | | enriched library, clone: 6530401A10 |
| | | | | product: Latexin, full insert sequence |
| Mus musulus | AK198791.1 | 56022968 | 100.00 | Mus musculus cDNA, clone: Y1G0129D08, |
| (Mouse) | | | | strand: minus, reference: ENSEMBL: |
| | | | | Mouse-Transcript-ENST: ENSMUST00 |
| | | | | 000058981, based on BLAT search |
| Mus musulus | AK187030.1 | 56011207 | 99.52 | Mus musculus cDNA, clone: Y0G0139J09, |
| (Mouse) | | | | strand: minus, reference: ENSEMBL: |
| | | | | Mouse-Transcript-ENST: ENSMUST00 |
| | | | | 000058981, based on BLAT search |
| Rattus | Y18435.2 | 6066618 | 94.33 | Rattus norvegicus Latexin gene, exons |
| norvegicus | | | | 1 to 6 |
| (Rat) | | | | |
| Rattus | Y18435.2 | 6066618 | 96.92 | |
| norvegicus | | | | |
| (Rat) | | | | |

The terms “treating”, and “treatment” and the like are used herein to generally mean obtaining a desired pharmacological and physiological effect. More specifically, the reagents described herein which are used to treat a subject with a tumor and metastatic disease generally are provided in a therapeutically effective amount to achieve any one or more of the following: inhibited tumor growth, reduction in tumor mass, loss of metastatic lesions, inhibited development of new metastatic lesions after treatment has started, or reduction in tumor such that there is no detectable disease (as assessed by e.g., radiologic imaging, biological fluid analysis, cytogenetics, fluorescence in situ hybridization, immunocytochemistry, colony assays, multiparameter flow cytometry, or polymerase chain reaction). The term “treatment”, as used herein, covers any treatment of a disease in a mammal, particularly a human.

By “therapeutically effective amount” is meant an amount of an agent, reagent, compound, composition or combination of reagents disclosed herein that when administered to a mammal is sufficient to be effective against the tumor.

By the term “tumor” is meant to include both benign and malignant growths or cancer. Thus, the term “cancer”, unless otherwise stated, can include both benign and malignant growths. Preferably, the tumor is malignant. The tumor can be a solid tissue tumor such as a melanoma, or a soft tissue tumor such as a lymphoma, a leukemia, or a bone cancer.

By the term “primary tumor” is meant the original neoplasm and not a metastatic lesion located in another tissue or organ in the patient's body.

By the terms “metastatic disease”, “metastases”, and “metastatic lesion” are meant a group of cells which have migrated to a site distant relative to the primary tumor.

### Latexin

Latexin is a 222 amino acid protein that has been largely studied in the central nervous system where it is primarily associated with the cortical margins during embryonic development (Arimatsu, Y. (1994) Neurosci Res 20, 131-135; Takiguchi-Hayashi, K. et al. (1995) Neuroreport 6, 281-283). In the adult peripheral nervous system, latexin is involved in the transmission of pain via nocioreceptors, because in latexin's absence, latency in response to pain was increased. (Jin, M., et al. (2006) Brain Res 1075, 117-121).

Latexin expression correlates with cell differentiation, as mature cells develop. In preliminary immunohistochemistry studies of latexin localization in the adult mouse brain, latexin was expressed in microglia cells. The Arimatsu group, which originally discovered latexin, reported its presence in mouse peritoneal mast cells, and localized it intracellularly to organelles distinct from mast cell granules and from typical lysosomes (Uratani, Y., et al. (2000) Biochem J 346 Pt 3, 817-826). However, latexin also appeared to reside in unique membraneous cytosolic particles whose function was unknown. A second study reported that latexin was constitutively expressed in mouse macrophages and that expression could be up-regulated by growth factors and pro-inflammatory stimuli (Aagaard, A. et al. (2005) Structure (Camb) 13, 309-317). The response of latexin to these stimuli was shown to work in concert with other types of protease inhibitors, and their target proteases. Thus, a role for latexin in the inflammatory response is shown. Chronic inflammation has been implicated in the etiology of a number of cancer types, and this raises the possibility that latexin may be associated with lymphomagenesis via its role in inflammation.

A strong homology exists between latexin and the tumor suppressor, tazarotene-induced gene 1 (TIG1). TIG1 is strongly down-regulated in a wide variety of tumors. Not only do latexin and TIG1 genetically map in close proximity; (and may accordingly represent two members of a common gene family), but they have strong structural similarities (Youssef, E. M., et al. (2004) Cancer Res 64, 2411-2417; Gautron, J., et al. (2001) J Biol Chem 276, 39243-39252; Hincke, M. T. et al. (2003) Connect Tissue Res 44 Suppl 1, 16-19). Latexin and TIG1 have been the subject of several molecular structure studies and the crystal structure of latexin has been resolved at high resolution by at least two groups independently (Pallares, I. et al. (2005) Proc Natl Acad Sci USA 102, 3978-3983; Mouradov, D. et al. (2005) Protein Eng Des Sel). The homology between latexin and TIG1 indicates that latexin is implicated in carcinogenesis.

### Latexin and Cancer

Many tumors, both solid and liquid, are now understood to derive from the malignant transformation of resident adult stem and progenitor cells. Latexin (LXN) was recently identified as a homeostatic regulator of the size of matopoietic stem cell populations in mice. It was found that the stem cell pool size was inversely related to quantitative latexin expression at both the transcription and protein levels. It was further found that the size of the population was influenced by latexin in a stem cell-autonomous manner, and acted through the concerted mechanisms of self-renewal and apoptosis, which were decreased and increased, respectively, by latexin abundance. Thus, latexin acts as a stopping mechanism on the expansion of the stem cell population.

Unrestrained stem cell expansion often results in mutations, genomic instability, and carcinogenesis. Accordingly, latexin expression patterns in stem and progenitor cells may influence the transformation of non-malignant cells into malignant cells. As latexin is a negative regulator of stem cell proliferation, it acts as a tumor suppressor by inhibiting stem and progenitor cell proliferation.

Several characteristics of latexin are consistent with the actions of a tumor suppressor. Latexin has about 30% sequence homology, and much greater structural homology, with tazarotene-induced gene 1 (TIG1), a tumor suppressor that is down-regulated via hypermethylation of its promoter in an extensive list of tumor types in humans. Further, latexin and TIG1 are closely linked genetically. Latexin was recently reported to be a TNF-responsive gene in human papillovirus-infected keratinocytes, suggesting latexin may contribute to the TNF-mediated suppression of cervical cancer development.

It has been found that latexin inhibits lymphoma cell growth by significantly increasing apoptosis, and that its anti-tumor activity is mediated via mechanisms that are unique from its canonical inhibition of carboxypeptidase A. Thus, latexin plays a functional role in tumor cell growth, and introduces a pathway for use in cancer treatment in patients.

Furthermore, latexin expression in cancer cells lines is absent or reduced compared to the latexin expression seen in normal cells. Gene silencing (such as silencing of tumor suppressors) and inappropriate gene activation (particularly of oncogenes) are common events in carcinogenesis. Both gene silencing and gene activation often occur through aberrant DNA methylation, which is accentuated during aging. Evidence from a variety of lymphoma and leukemia cell lines, as well as from primary cells from patients with these diseases, shows that latexin expression is almost universally absent or significantly reduced from that of normal stem and progenitor cells. Moreover, genetic polymorphisms in the latexin promoter region, in concert with methylation patterns of the CpG island, affect transcription. Treatment with a de-methylating agent at least partially restores latexin expression in a variety of tumor cell lines. Finally, when latexin expression was re-initiated ectopically in two lymphoma cell lines using a retroviral expression vector, their growth, both in vitro and in vivo, was significantly blunted.

Lxn expression in stem cells is inversely proportional to the size of the population in mice, and thus acts as a negative regulator through cell-intrinsic mechanisms. The observation that low Lxn expression in hematopoietic cells was associated with increased replication shows Lxn was down-regulated in malignancies with high proliferative rates. Latexin is either not expressed or is strongly down-regulated in a variety of leukemias and lymphomas. Based on these findings and the close structural and genetic linkage of latexin with TIG1, latexin act as a tumor suppressor. Thus, manipulation of latexin levels in malignant cells would alter their growth characteristics. Accordingly, two lymphoma cell lines in which Lxn expression was absent were infected with a Lxn expression vector. It was found that re-initiation of Lxn expression dramatically reduced cell growth in vitro and in vivo. These results indicate the level of Lxn expression is functionally related to both normal and malignant cell growth.

The possible mechanisms by which ectopic Lxn expression may suppress growth of lymphoma cells was investigated, and found increased apoptosis is the major mechanism causing growth inhibition. This finding is consistent with the study of normal hematopoietic cells, in which high latexin content is associated with more apoptotic cells and thus fewer stem cells. A only known function of latexin is as the sole carboxypeptidase A inhibitor in mammals. Thus, other carboxypeptidase A inhibitors might mimic the anti-tumor effects of latexin. CPA inhibitors from potato tubers have been shown to be an effective inhibitor of mammalian CPA, and it was found that it freely entered A20 lymphoma cells, but did not have suppressive growth effects via apoptosis, irrespective of dose. Thus, latexin is responsible for enhanced apoptosis, and previously unknown intracellular functions of latexin account for its anti-tumor effects.

As with many tumor suppressors, it was found that CpG dinucleotides in the regulatory regions of the latexin gene were methylated. Most human leukemia and lymphoma cell lines tested showed a strong pattern of methylation involving most, if not all, of the 15 CpG dinucleotides in the latexin promoter. This, in turn, led to a loss of gene expression that was partially reversible. When the cell lines were treated with the demethylating reagent, 5-aza-2′-deoxycytidine, latexin expression was re-initiated or up-regulated. Moreover, a detailed analysis of CD34+CD38− cells from two AML patients revealed selective methylation in potentially pivotal regions of the latexin promoter that may impede the binding of transcription factors. Candidates were identified by sequence homologies between transcription factor binding and latexin promoter sequence, and all are involved in tumorigenesis. (Pirkkala L. et al., FASEB J 1999; 13:1089-98; Sistonen L. et al., Mol Cell Biol 1992; 12:4104-11; Vaclavicek A. et al., Breast Cancer Res Treat 2007; 106:205-13; Lo Y L. et al., Carcinogenesis 2007; 28:1079-86; O′Neil J, Oncogene 2007; 26:6838-49; Turco M C., Leukemia 2004; 18:11-7; Gilmore T D., Oncogene 2004; 23:2275-8). HSF2, V-Myb, and REL/NF-κB are of particular interest due to their mediation of hematopoietic malignancies. Thus, hypermethylation of the CpG island in the Lxn promoter coupled with aberrant transcription factor binding may, singly or in concert, contribute to silencing or down-regulation of latexin expression in malignant cells.

### Lymphoma

The majority of B cell lymphoma arises in humans after the age of 50, and the incidence of this type of cancer is increasing in the United States and other parts of the world (Lichtman, M. A. (2008) Oncologist 13, 126-138). The World Health Organization classification lists nineteen types of B cell lymphomas. The cell in the B cell developmental pathway in which the transforming event(s) occurs that entrains lymphoma development is unknown in many of the lymphoma sub-types (Gniadecki, R. (2004) Archives of Dermatology 140, 1156-1160). Many lymphomas arise from transforming events in early precursors, including the lympho-hematopoietic stem cells in the bone marrow. Other lymphomas arise from more differentiated cells in the pathway, by re-acquiring proliferative capacities and self-renewal properties reminiscent of the progenitors from which they were derived.

Two models, a “horizontal” and a “vertical”, are proposed for lymphomagenesis. Historically, the development of lymphoma has been thought of as “horizontal”, i.e., a given type of lymphoma reflected the properties of a normal, partially, or wholly differentiated cell in the B or T cell pathway, from which it was derived by acquiring unlimited proliferative capacity and resistance to apoptosis. Its functional properties were a “frozen” reflection of those of the cell from which it was derived, hence the term “horizontal” derivation (Zutter M et al., The biology of low-grade malignant lymphoma. In: Cancellos G, Lister T, Sklar J, eds. The Lymphomas. Philadelphia: WB Saunders; 1998:337-351). The cell in the B cell developmental pathway in which the transforming events occur that entrains lymphoma development is unknown in many of the lymphoma sub-types (Gniadecki R. et al., Neoplastic Stem Cells in Cutaneous Lymphomas. Archives of Dermatology. 2004; 140:1156-1160). Many lymphomas seemingly arise from transforming events in precursors, including the lympho-hematopoietic cells in the bone marrow. In this contrasting “vertical” model of carcinogenesis, a cancer stem cell arises and passes on the transforming event to its progeny, which at some point in the vertical developmental scheme, begin to express aberrant growth and differentiation characteristics of the tumor.

The “vertical” model of lymphomagenesis illustrates the close relationship between the myeloid leukemias and the lymphoid lymphomas. Within the immuno-hematopoietic system, which originates with pluripotent stem cells in the bone marrow, neoplastic events subverting both arms of differentiation toward carcinogenesis use similar, if not identical, mechanistic modifications. The myeloid and lymphoid lineages, particularly the respective end cells, are considerably different. However, a number of the mature myeloid lineage cells are engaged in the same comprehensive function as lymphoid cells (i.e., as host defense mechanisms). Several myeloid lineage cells function closely with lymphoid cells for this purpose, including antigen presenting cells and facilitator cells.

There are several aspects of the “horizontal” theory fail to adequately account for clinical aspects of lymphomas. First is the heterogeneity found within many cases of lymphoma. If the primary lesion occurred in a cell at a specific point during lymphoid development and the clonal derivative developed horizontally, one would expect that cells in the clone would be homogeneous. This is often not the case. The probability of simultaneous neoplastic events in cells at different steps along lymphoid development is highly unlikely. The presence of composite lymphomas in both the B and T cell arms, respectively, sometimes with precisely the same genetic rearrangements in the various components, is better explained by a stem or progenitor cell etiology and extending “vertically”. Cells further down the developmental pathway from the cell harboring the prima facie transforming event are each potentially capable of giving rise to a tumor with characteristics unique to its stage of development, including cell surface markers, immunoglobulin gene rearrangements, etc. Thus, the final phenotype of a lymphoma (or of any tumor) does not necessarily reflect the phenotype of the cell that undergoes the initial malignant transformation. Such a cell may be so early in the developmental pathway as to have no phenotype coordinate with B or T cell development. Although there is recently described evidence for this in B lymphomas (van den Berg, A., et al. (2002) Blood 100, 1425-1429), further evidence comes from the cutaneous T lymphomas where the range of subtypes in composite tumors can be strikingly large (Gniadecki, R., et al. (2003) Blood 102, 3797-3799; Berg, K. D., et al. (2001) N Engl J Med 345, 1458-1463; Assaf, C., et al. (2003) Br J Haematol 120, 488-491).

The refractoriness of some lymphomas to chemotherapy and radiation provides further support for a stem or progenitor cell origin of some lymphomas. These cell populations are often mitotically quiescent and thus inaccessible to agents specific to the cell cycle. The vertical model accounts for surviving “lymphoma stem cells”, often residing in the bone marrow, which give rise to the relapses commonly seen in patients with lymphomas.

The present invention favors a “vertical” mode of lymphomagenesis and therefore focuses on early progenitors in bone marrow and the factors that may either predispose them, or transform them, to malignancy. In particular, the present invention uses the lympho-hematopoietic stem cell regulating abilities of latexin, which acts as a negative regulator of the size of the stem cell population in the bone marrow (Liang, Y., et al. (2007) Nat Genet. 39, 178-188). Latexin acts as a brake on stem cell self-renewal and thus pool size. Thus, in its absence, the resulting unrestrained stem cell proliferation would create conditions rife for genetic lesions, genetic instability, and malignant transformation. Latexin acts as a tumor suppressor in lymphoma such that mouse and human B cell lymphoma cell lines either express no latexin expression or greatly reduced levels.

A further link between latexin and lymphoma, as well as with other cancers, originated from the role of latexin in regulating hematopoietic stem cell population size in bone marrow. Latexin quantitatively regulates stem cells by diminishing self-renewal and enhancing stem cell apoptosis (Liang, Y. et al. (2007) Nat Genet. 39, 178-188). These two control pathways are frequently dysregulated in malignancy.

In one aspect of the invention, the methods and compositions disclosed herein can be used to inhibit or slow the progression of malignancies through the modulation of latexin. These malignancies can be solid or soft tissue tumors. Soft tissue tumors include bone cancers, lymphomas and leukemias. Another aspect of the invention uses the methods and compositions to inhibit or prevent metastases or metastatic progress.

Agents which modulate latexin can be used alone or in combination with other cancer modalities, such as but not limited to chemotherapy, surgery, radiotherapy, hyperthermia, immunotherapy, hormone therapy, biologic therapy (e.g., immune effector mechanisms resulting in cell destruction, cytokines, immunotherapy, interferons, interleukin-2, cancer vaccine therapy, and adoptive therapy), and drugs to ameliorate the adverse side effects of such cancer modalities.

### Cancer Treatment

The term cancer embraces a collection of malignancies with each cancer of each organ consisting of numerous subsets. Typically, at the time of cancer diagnosis, “the cancer” consists in fact of multiple subpopulations of cells with diverse genetic, biochemical, immunologic, and biologic characteristics.

Preferred cancers include but are not limited to melanomas (e.g., cutaneous melanoma, metastatic melanomas, and intraocular melanomas), prostate cancer, lymphomas (e.g., cutaneous T-cell lymphoma, mycosis fungoides, Hodgkin's and non-Hodgkin's lymphomas, and primary central nervous system lymphomas), leukemias (e.g., pre-B cell acute lymphoblastic leukemia, chronic and acute lymphocytic leukemia, chronic and acute myelogenous leukemia, adult acute lymphoblastic leukemia, mature B-cell acute lymphoblastic leukemia, prolymphocytic leukemia, hairy cell leukemia, and T-cell chronic lymphocytic leukemia), and metastatic tumors which exhibit these proteins on the cell surface. These cancers may include, but are not limited to breast cancer, cervical cancer, lung cancer, and cancer of the gastrointestinal tract. Although mycosis fungoides (MF), Sézary syndrome, reticulum cell sarcoma of the skin and several other cutaneous lymphocytic dyscrasias were once considered separate conditions, they are now recognized as different clinical presentations of cutaneous T-cell lymphoma (CTCL) and thus included in the term. See Lynn D. Wilson et al., “Cutaneous T-Cell Lymphomas,” IN CANCER: PRINCIPLES & PRACTICE OF ONCOLOGY 2220-2232 (Vincent T. DeVita, Jr. et al., editors, 5th ed. 1997); Bank et al., 1999, J. Cutan. Pathol., 26(2): 65-71.

### Metastatic Disease

Once a tumor is diagnosed in a patient, an important concern is whether the tumor has progressed and spread to the regional lymph nodes and to distant organs. Most cancer deaths result from metastases that are resistant to conventional cancer therapies. Metastases can be located in different organs and in different regions of the same organ, making complete eradication by surgery, radiation, drugs, or biotherapy nearly impossible.

Thus, also contemplated for treatment with the methods, combination therapies and compositions disclosed herein, is the treatment of metastatic cancer. Cancers typically begin their growth in only one location in the tissue of origin. As the cancer progresses, the cancer may migrate to a distal location in the patient. For example, a cancer beginning in the prostate may migrate to the lung. Other locations common for metastatic disease and that are contemplated herein include metastatic cancer to the brain, lung, liver, and bone.

There are essential steps in the formation of metastasis in all tumors. The steps include the following:
(1) After neoplastic transformation, progressive proliferation of neoplastic cells supported by the organ/tissue environment in which the neoplasm is located.
(2) Neovascularization or angiogenesis of the tumor for further growth beyond 1 to 2 mm in diameter.
(3) Down-regulation of expression of cohesive molecules wherein the cells have increased motility or ability to detach from the primary lesion.
(4) Detachment and embolization of single tumor cells or cell aggregates, with the vast majority of these cells being rapidly destroyed.
(5) Once tumor cells survive the detachment and embolization step, they must go on to proliferate within the lumen of the blood vessel. The cells will then go on to extravasate into the organ parenchyma by mechanism similar to those operative during invasion.
(6) Tumor cells with the appropriate cell surface receptors can respond to paracrine growth factors and hence proliferate in the organ parenchyma.
(7) Tumor cell evasion of host defenses (both specific and nonspecific immune responses).
(8) For a metastasis to proliferate beyond 1 to 2 mm in diameter, the metastases must develop a vascular network.

Thus, if a primary tumor is given enough time to go through these steps, it will form metastatic lesions at a site or sites distant to the primary tumor. The reagents, methods, and combination therapies disclosed inhibit or prevent one or more of these steps in the metastatic process. For additional details on the mechanism and pathology of tumor metastasis, see Isaiah J. Fidler, “Molecular Biology of Cancer: Invasion and Metastasis,” in CANCER: PRINCIPLES & PRACTICE OF ONCOLOGY 135-152 (Vincent T. DeVita et al., editors, 5th ed., 1997).

Many treatments exist for cancers. The particular cancer therapy or combination of therapy modalities used to treat a cancer depend greatly on the type of cancer, its stage, the patient (e.g., weight, sex, age, health, prior cancers, and the like), and where the patient is in therapy (e.g., first treatment, in blast crisis, refractive to initial treatments, cancer relapse, or a second cancer perhaps induced by the treatment of the first cancer months or years before). Therefore, physicians will frequently have to combine a variety of treatment modalities which will best suit the needs of the patient in combating the disease and the patient's self-determination of quality of life. Treatment modalities include but are not limited to surgery, radiation therapy, chemotherapy, biologic therapy (e.g., cytokines, immunotherapy, and interferons), hormone therapies, and hyperthermia.

Conventional chemotherapy can be further broken down into hormone therapies (e.g., antiestrogens, aromatase inhibitors, gonadotropin-releasing hormone analogues, and anti-androgens), anti-tumor alkylating agents (e.g., mustards, nitrosoureas, tetrazines, and aziridines), cisplatin and its analogues, anti-metabolites (e.g., methotrexate, antifolates, 5-fluoropyrimidines, cytarabine, azacitidine, gemcitabine, 6-thipurines, and hydroxyurea), topoisomerase interactive agents, antimicrotubule agents (e.g., vinca alkaloids, taxanes, and estramustine), differentiating agents (e.g., retinoids, vitamin D3, polar-apolar compounds, butyrate and phenylactetate, cytotoxic drugs, cytokines, and combinations thereof), and other chemotherapeutic agents such as fludarabine, 2-chlorodeoxyadenosine, 2′-deoxycoformycin, homoharringtonine (HHT), suramin, bleomycin, and L-asparaginase.

One aspect of the invention contemplates the use of latexin to treat lymphomas. Lymphomas contemplated for treatment by these combination therapies include T-cell lymphomas such as but not limited to cutaneous T-cell lymphoma (CTCL), T-cell non-Hodgkin's lymphoma, peripheral T-cell lymphomas, anaplastic large-cell lymphoma, anti-immunoblastic lymphoma, and precursor T-LBL. Treatment of lymphomas is again dependent on the subject being treated, the type of disease and its stage. Existing treatment modalities for leukemias and lymphomas are described generally in CANCER: PRINCIPLES & PRACTICE OF ONCOLOGY (Vincent T. DeVita et al., editors, 5th ed., 1997). Also contemplated for treatment are B-cell lymphomas (e.g., follicular lymphoma, diffuse large B-cell lymphoma, mantle cell lymphoma, B-CLL/SLL, immunocytoma/Waldenstrom's, and MALT-type/monocytoid B cell lymphoma). Also contemplated are the treatment of pediatric lymphomas such as Burkitt's lymphoma, diffuse large B-cell lymphoma, follicular lymphoma, precursor B-LBL, precursor T-LBL, anaplastic large cell lymphoma, and peripheral T-cell lymphoma.

Common drug combinations for use in treating lymphomas include but are not limited to CHOP (i.e., cyclophosphamide, doxorubicin, vincristine, and prednisone), GAP-BOP (i.e., cyclophosphamide, doxorubicin, procarbazine, bleomycin, vincristine, and prednisone), m-BACOD (i.e., methotrexate, bleomycin, doxorubicin, cyclophosphamide, vincristine, dexamethasone, and leucovorin), ProMACE-MOPP (i.e., prednisone, methotrexate, doxorubicin, cyclophosphamide, etoposide, leucovorin with standard MOPP), ProMACE-CytaBOM (prednisone, doxorubicin, cyclophosphamide, etoposide, cytarabine, bleomycin, vincristine, methotrexate, and leucovorin), and MACOP-B (methotrexate, doxorubicin, cyclophosphamide, vincristine, prednisone, bleomycin, and leucovorin). For relapsed aggressive non-Hodgkin's lymphoma the following chemotherapy drug combinations may be used with the antibodies and drug combinations described herein: IMVP-16 (i.e., ifosfamide, methotrexate, and etoposide), MIME (i.e., methyl-gag, ifosfamide, methotrexate, and etoposide), DHAP (i.e., dexamethasone, high dose cytarabine, and cisplatin), ESHAP (i.e., etoposide, methylprednisone, high dosage cytarabine, and cisplatin), CEFF(B) (i.e., cyclophosphamide, etoposide, procarbazine, prednisone, and bleomycin) and CAMP (i.e., lomustine, mitoxantrone, cytarabine, and prednisone). See Margaret A. Shipp, et al., “Non-Hodgkin's Lymphomas,” in CANCER: PRINCIPLES & PRACTICE OF ONCOLOGY 2165-2220 (Vincent T. DeVita et al., editors, 5th ed., 1997).

Treatment for salvage chemotherapy used for certain lymphomas such as for relapsed, resistant Hodgkin's Disease include but are not limited to VABCD (i.e., vinblastine, doxorubicin, dacarbazine, lomustine and bleomycin), ABDIC (i.e., doxorubicin, bleomycin, dacarbazine, lomustine, and prednisone), CBVD (i.e., lomustine, bleomycin, vinblastine, dexamethasone), PCVP (i.e., vinblastine, procarbazine, cyclophosphamide, and prednisone), CEP (i.e., lomustine, etoposide, and prednimustine), EVA (i.e., etoposide, vinblastine, and doxorubicin), MOPLACE (i.e., cyclophosphamide, etoposide, prednisone, methotrexate, cytaravine, and vincristine), MIME (i.e., methyl-gag, ifosfamide, methotrexate, and etoposide), MINE (i.e., mitoquazone, ifosfamide, vinorelbine, and etoposide), MTX-CHOP (i.e., methotrexate and CHOP), CEM (i.e., lomustine, etoposide, and methotrexate), CEVD (i.e., lomustine, etoposide, vindesine, and dexamethasone), CAVP (i.e., lomustine, melphalan, etoposide, and prednisone), EVAP (i.e., etoposide, vinblastine, cytarabine, and cisplatin), and EPOCH (i.e., etoposide, vincristine, doxorubicin, cyclophosphamide, and prednisone). See, e.g., Vincent T. DeVita et al., “Hodgkin's Disease,” in CANCER: PRINCIPLES & PRACTICE OF ONCOLOGY 2242-2283 (Vincent T. DeVita et al., editors, 5th ed., 1997).

Thus, one aspect of the invention contemplates the use of agents with modulate latexin expression to be used to inhibit lymphoma progression in a subject and/or metastasis of a lymphoma. The reagents can be used either alone, or in combination with other lymphoma treatments as discussed herein.

Another aspect of the invention contemplates inhibiting melanoma growth and/or inhibiting growth or spread of melanoma metastases. The methods and compositions are contemplated for but not limited to treating cutaneous melanomas, metastatic melanomas and intraocular melanomas. Conventional therapies for treating these melanomas are known in the art. See e.g., Anthony P. Albino et al., “Molecular Biology of Cutaneous Malignant Melanoma,” IN CANCER: PRINCIPLES & PRACTICE OF ONCOLOGY 1935-1947 (Vincent T. DeVita et al., editors, 5th ed., 1997); Charles M. Balch et al., “Cutaneous Melanoma,” IN CANCER: PRINCIPLES & PRACTICE OF ONCOLOGY 1947-1994; and Jose A. Sahel et al., “Intraocular Melanoma,” IN CANCER: PRINCIPLES & PRACTICE OF ONCOLOGY 1995-2012.

For example, for treatment of metastatic melanoma, use of surgery, isolated limb perfusion, regional chemotherapy infusion (with e.g., decarbazine or cisplatin), radiation therapy, immunotherapy (e.g., treatment with antibodies against GD2 and GD3 gangliosides), intralesional immunotherapy, systemic chemotherapy, hyperthermia, systemic immunotherapy, tumor vaccines, or combinations thereof can be further combined with latexin and/or latexin modulators.

Another aspect of the invention provides for the treatment of certain leukemias using agents which modulate latexin in combination with conventional treatment modalities for the leukemia to be treated.

Traditional treatment for acute myelogenous leukemia (AML) includes but is not limited to anthracycline/cytarabine-based induction regimens, intensive post-remission therapy such as bone marrow transplant (BMT) or high-dose (HD) cytarabine. Traditional treatment for acute promyelocytic leukemia includes but is not limited to retinoic acid and anthracycline/cytarabine-based treatment. Patients may also be administered a cryoprecipitate or fresh frozen plasma to maintain fibrinogen levels of greater than 100 mg/dL. Platelet transfusions may also be necessary to maintain a daily platelet count in a human of >50,000 μL. Traditional treatment modalities for acute lymphoblastic leukemia (ALL) includes four or five drug induction regimens using anthracyclines, cyclophosphamide, asparaginase or a combination in addition to vincristine and prednisone. Alternatively, the physician may opt to use an intensive consolidation therapy based on cytarabine combined with anthracyclines, epidophillotoxins, or anti-metabolites in combination with the immunoglobulin compositions described herein. Yet another aspect may be the use of protracted maintenance therapy using oral methotrexate combined with mercaptopurine and the subject immunoglobulins. Another alternative contemplates the use of prophylactic intrathecal chemotherapy (with or without cranial radiotherapy) for CNS prophylaxis in combination with the subject immunoglobulins. For additional information on the therapy modalities for treating leukemia that can be used in combination with the instant invention, see Issa Khouri et al., “Molecular Biology of Leukemias,” IN CANCER: PRINCIPLES & PRACTICE OF ONCOLOGY 2285-2293 (Vincent T. DeVita et al., editors, 5th ed., 1997); David A. Scheinberg et al., “Acute Leukemias,” in CANCER: PRINCIPLES & PRACTICE OF ONCOLOGY 2293-2321; and Albert B. Deisseroth et al., “Chronic Leukemias,” in CANCER: PRINCIPLES & PRACTICE OF ONCOLOGY 2321-2343.

Treatment of metastases can be with the compositions, combination therapies and methods described herein by themselves or in combination with other cancer treatment modalities depending on the site of the metastases and the primary tumor from which the metastases originates. The most common sites for tumors to metastasize are brain, lung, liver, bone, malignant pleural and pericardial effusions, and malignant ascites.

### Formulations and Methods of Administration

The agents of interest discussed above preferably are administered in a physiologically acceptable carrier to a subject. The agents may be administered in a variety of ways including but not limited to parenteral administration, including subcutaneous (s.c.), subdural, intravenous (i.v.), intramuscular (i.m.), intrathecal, intraperitoneal (i.p.), intracerebral, intraarterial, or intralesional routes of administration, localized (e.g., surgical application or surgical suppository), and pulmonary (e.g., aerosols, inhalation, or powder) and as described further below. Preferably, the agents are administered intravenously or orally.

Preferably, the agents which modulate latexin expression are administered intravenously or intramuscularly every few days. Alternatively, preferably the agents may be administered orally.

Depending upon the manner of introduction, the agents may be formulated in various ways. Preferably, the agent may be formulated for parenteral administration in a suitable inert carrier, such as a sterile physiological saline solution. The dose administered will be determined by route of administration.

For parenteral administration, the agents of the invention can be administered as injectable dosages of a solution or suspension of the substance in a physiologically acceptable diluent with a pharmaceutical carrier, which can be a sterile liquid such as water and oils with or without the addition of a surfactant. Other acceptable diluents include oils of animal, vegetable, or synthetic origin, for example, peanut oil, soybean oil, and mineral oil. In general, glycols such as propylene glycol or polyethylene glycol (PEG) are preferred liquid carriers, particularly for injectable solutions. The antibodies and immunoglobulins of this invention can be administered in the form of a depot injection or implant preparation, which can be formulated in such a manner as to permit a sustained release of the active ingredient(s). Administration of other cancer therapeutic agents can occur prior to, concurrent with, or after administration with the agents. Administration of the subject immunoglobulins can occur before, during or after surgical treatment, radiotherapy, hormone therapy, immunotherapy, hyperthermia, or other cancer treatment modality. Administration of the subject agents can occur daily, weekly, or monthly as needed. Preferably, the immunoglobulins are administered weekly for one or more weeks.

Pharmaceutical compositions comprising these agents can also include, if desired, pharmaceutically acceptable, non-toxic carriers or diluents, which are vehicles commonly used to formulate pharmaceutical compositions for animal or human administration. The diluent is selected so as not to affect the biological activity of the combination. Examples include but are not limited to distilled water, physiological phosphate-buffered saline, Ringer's solutions, dextrose solution, and Hank's solution.

The agents of the invention can be formulated into preparations for injections by dissolving, suspending or emulsifying them in an aqueous or non-aqueous solvent, such as vegetable or other similar oils, synthetic aliphatic acid glycerides, esters of higher aliphatic acids or propylene glycol. The formulations may also contain conventional additives, such as solubilizers, isotonic agents, suspending agents, emulsifying agents, stabilizers and preservatives.

Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate)) as described by Langer et al., J. Biomed. Mater. Res. 15: 167-277 (1981) and Langer, Chem. Tech. 12: 98-105 (1982) or poly(vinylalcohol)), polylactides (U.S. Pat. No. 3,773,919), copolymers of L-glutamic acid and gamma ethyl-L-glutamate (Sidman et al., Biopolymers 22: 547-556, 1983), non-degradable ethylene-vinyl acetate (Langer et al., supra), degradable lactic acid-glycolic acid copolymers such as the LUPRON DEPOT™ (i.e., injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity. Rational strategies can be devised for immunoglobulin stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, developing specific polymer matrix compositions, and the like.

The agents of this invention can be administered in a sustained release form, for example a depot injection, implant preparation, or osmotic pump, which can be formulated in such a manner as to permit a sustained release of the active ingredient. Implants for sustained release formulations are well-known in the art. Implants are formulated as microspheres, slabs, etc. with biodegradable or non-biodegradable polymers. For example, polymers of lactic acid and/or glycolic acid form an erodible polymer that are well-tolerated by the host. The implant is placed in proximity of a solid tumor for example, so that the local concentration of active agent is increased at that site relative to the rest of the body.

Although the present invention has been described in detail with reference to example below, it is understood that various modifications can be made without departing from the spirit of the invention, and would be readily known to the skilled artisan.

### EXAMPLE

### Example 1

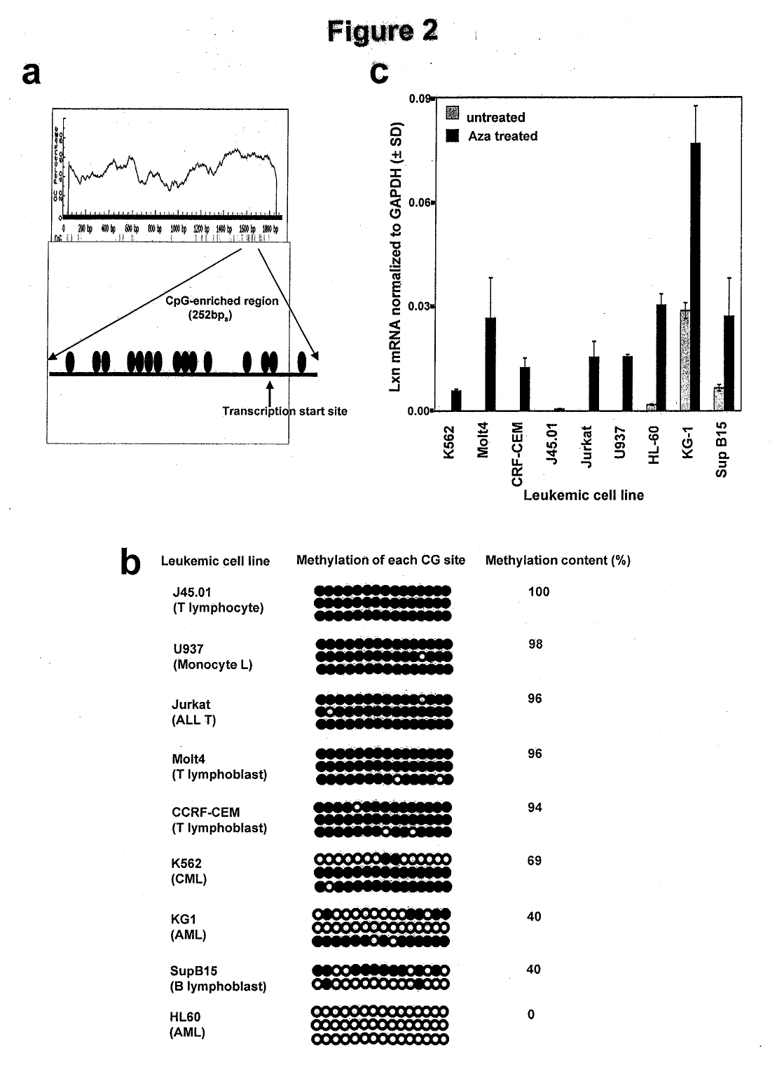

It was determined by quantitative real-time PCR, mRNA expression of Lxn in tumor cell lines, bone marrow and peripheral blood CD34+ cells from lymphoma and leukemia patients and normal individuals (FIG. 2a). The patients tested included acute myeloid leukemia (AML), T cell pro-lympho leukemia (PCTL), plasma cell leukemia (PCL), acute T cell lymphoma (ATLL) and acute lymphoid leukemia (ALL, preB phenotype). The normal samples were derived from cord blood (CB) and young (27 and 28 years) and old (83 and 97 years) adults. Compared with normal primitive hematopietic cells, Lxn mRNA expression was decreased to at least one-third in primary malignant CD34+ cells and significantly diminished in HL-60, KG-1 and SupB15 cell lines. Its expression was completely lost in most of leukemic lines, including K562, Molt4, CRF-CEM, J45.01, Jurkat, U937 and K562. latexin protein expression was also tested in all samples using Western blotting and nearly identical results were found in leukemic cell lines compared to normal young marrow CD34+ cells (YBM) (FIG. 2b). Quantification of latexin protein in CD34+ cells of all human normal and leukemic samples compiled to date are plotted in FIG. 2c, and show a significant decrease of latexin expression in malignant cells (P=0.03).

FIG. 2 shows that latexin expression is down-regulated or absent in a variety of mouse and human lymphoma and leukemia cell lines, and in primary malignant cells from patients with these diseases. Latexin was ectopically expressed in the mouse lymphoma cell lines WEHI 231 and A20 using an Sf beta-based retroviral expression vector. As a control the same vector backbone was used, which lacked the latexin gene. Both vectors contained green fluorescent protein (GFP) as a marker of infected cells selectable by flow cytometric cell sorting. The two mouse lymphoma cell lines were retrovirally infected in vitro, GFP positive cells were purified by cell sorting and their growth patterns in vitro (both A20 and WEHI 231) and in vivo (A20) were determined.

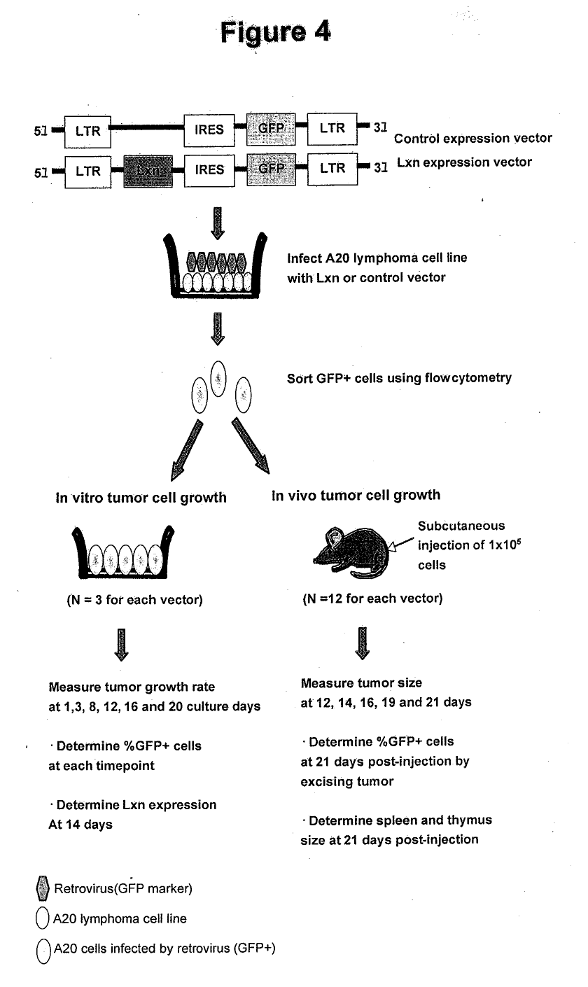

FIG. 4 shows results of a representative experiment in which the same A20 cells were tested for growth both in vitro and in vivo, and it shows the potent effects of latexin expression on inhibiting tumor growth under both growth conditions. FIG. 4a shows results of latexin expression on in vitro growth. A20 cell cultures were counted and split every 3-4 days to keep the cells at a relatively constant cell density and to provide fresh medium. Cultures of A20 cells infected with the latexin expression vector contained only about half the number of cells after day 3 as compared with A20 cells infected with the control (empty) vector or uninfected control cells (A20 control). An aliquot of cells was collected at each of the time-points where cells were quantified to determine the durability of GFP (and presumably latexin) expression. FIG. 4b shows that the fraction of GFP+ cells remained at 90-100% throughout the 20 days of culture in both the latexin vector- or control vector-infected cells. FIG. 4c shows that at day 15 of culture neither uninfected A20 cells nor A20 cells infected with the control (empty) vector expressed detectable latexin protein, whereas in the Western blot a strong latexin band was evident in lysate of cells infected with the latexin vector (upper band). The blots were stripped and re-probed for actin (bottom band) to show that the amount of lysate loaded per lane was approximately equal for the three cell types.

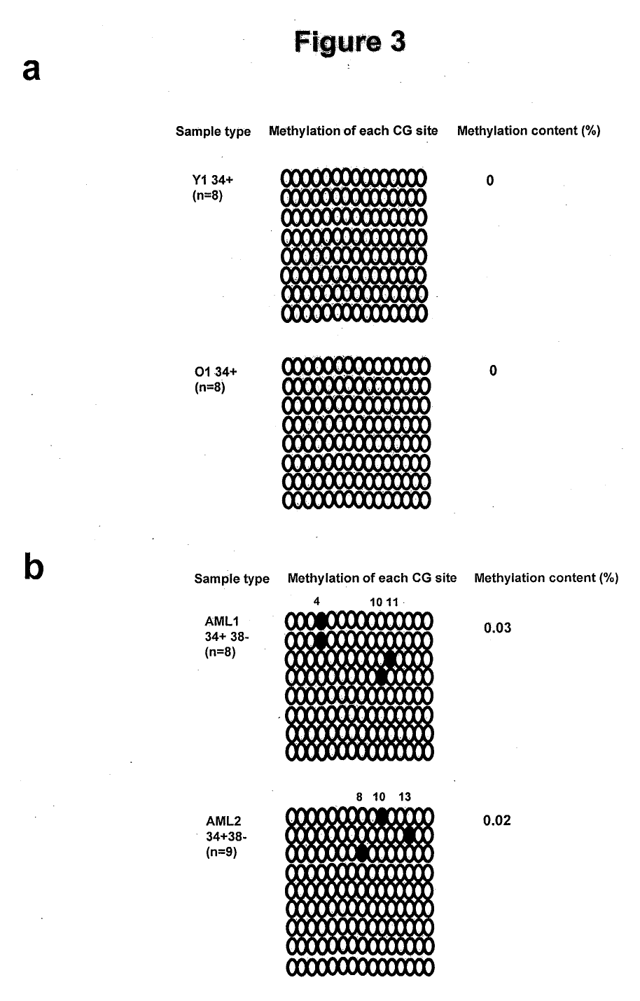

FIG. 4d shows the results of the in vivo arm of the experiment (refer to FIG. 3), and demonstrates the dramatic reduction in tumor size caused by ectopic latexin expression in A20 cells. One hundred thousand cells were injected in a 50 μl bolus subcutaneously in the shaved flank of Balb/cJ mice given 3.0 Gy of gamma radiation 4 hrs prior. Lymphomas were detectable by palpation 10-12 days post-injection and all three dimensions of tumors were measured with calipers on days 12, 14, 16, 19, and 21. Tumor volume was calculated by multiplying Lx W×H. The same person performed tumor measurements at each time-point and did not know which group the animals came from; they were measured blind. As soon as tumors were measurable, the latexin-expressing cells caused significantly smaller tumors (P<0.05). By day 21, the tumors in the latexin vector-infected group were 40% of the volume of tumors in the other two groups (P<0.01). FIG. 4e depicts tumor growth on a semi-log basis where tumor volume on the y-axis is on a log scale. The growth rate (in cubic volume) of tumors originating from latexin vector-infected A20 cells or empty vector-infected cells is roughly equivalent, as is evident by the roughly parallel plots with similar slopes. The dramatic difference in tumor size between the two groups owes to a fundamental difference at tumor inception and suggests that latexin may prevent tumor formation completely with smaller sized inocula, and is potentially important at the inception of lymphoma development.

At day 21 of the experiment reported in FIG. 4, the mice were euthanized and the lymphomas were excised, and single cell suspensions were made of each to determine the fraction of tumor cells expressing GFP. FIG. 4f shows that virtually all of the tumor cells in the latexin and control vector groups were GFP+. More to the point, Western blots confirmed strong expression of latexin as in the cells analyzed after 15 days in culture (FIG. 4). Thus, the reduction in tumor growth was due to durable latexin expression in the tumor cells themselves. Host animals were necropsied for evidence of gross metastases to spleen, thymus and liver. No evident tumors were found in any of the treatment groups. Similarly, flow cytometry detected no GFP+ cells in these anatomical sites.

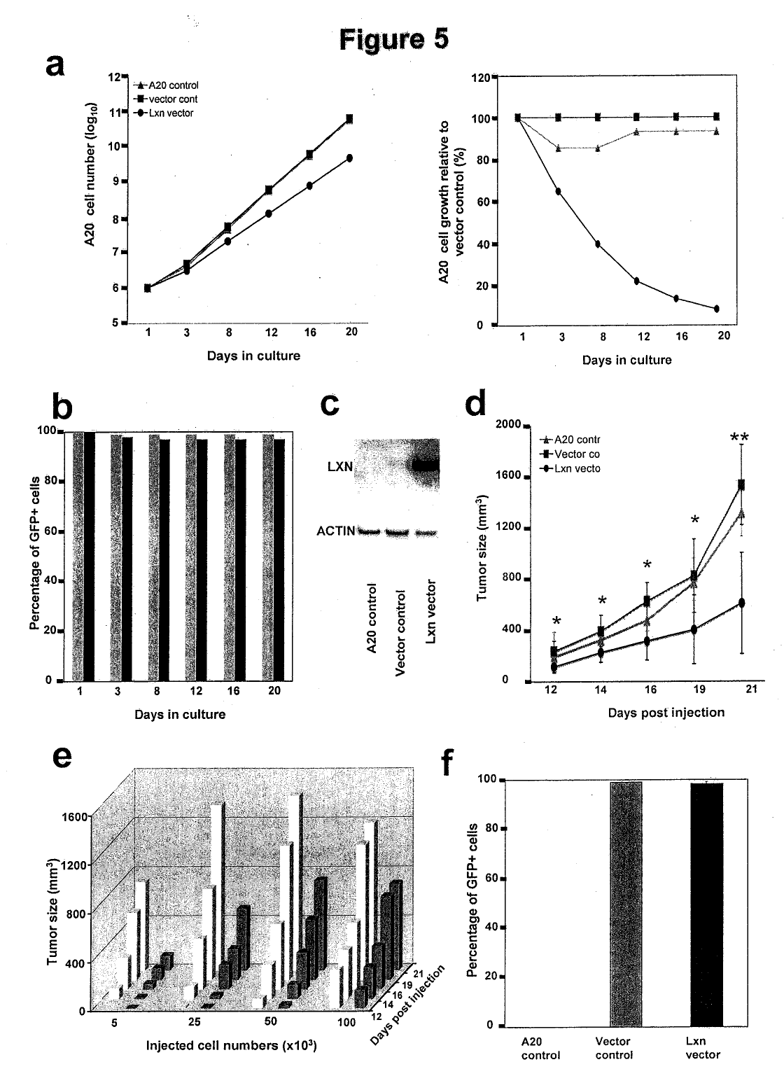

FIG. 5 shows the results of replicate experiments testing ectopic latexin expression on the growth in vitro of A20 and WEHI 231 cells. Similar to the data presented in FIG. 4a, ectopic latexin expression in both A20 and WEHI 231 cells reduced cell growth by about half, a reduction nearly identical to what was found in vivo. Four independent in vitro experiments of this type have been carried out for both A20 and WEHI cells.

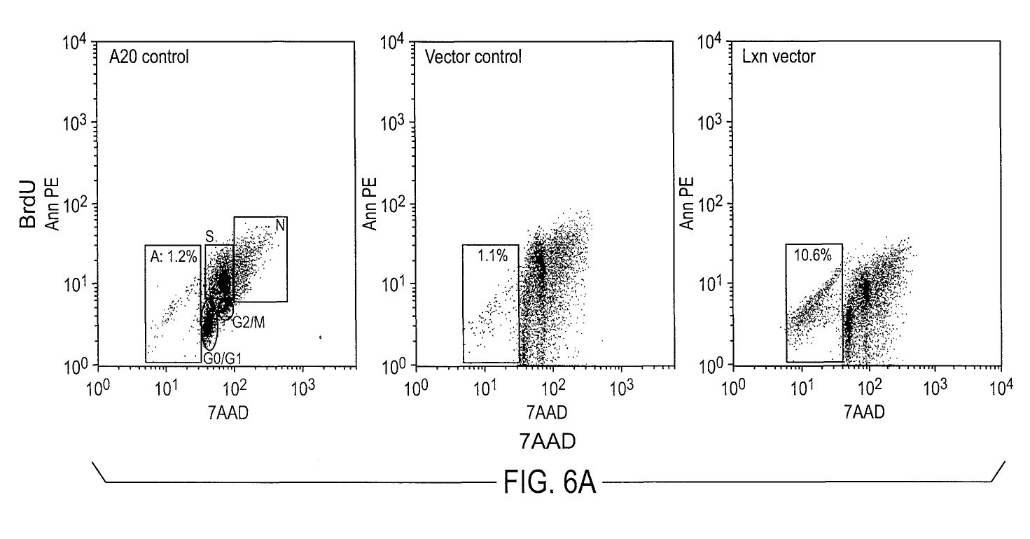

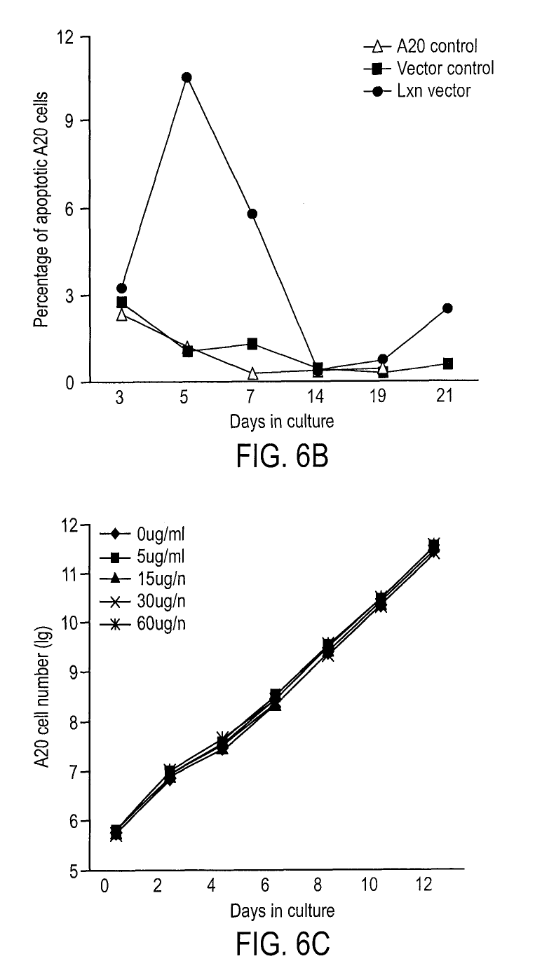

The only known function of latexin is its role as the sole carboxypeptidase A inhibitor (CPI) in mammalian cells. Therefore, to test whether or not the suppressive effect of ectopic latexin expression on the growth of A20 and WEHI 231 lymphoma cells is due to its canonical CPI activity, the following experiments were carried out. Potato CPI, a 39 amino acid protein, was added to cultures of A20 and WEHI 231 cells in a series of concentrations ranging from 0 to to 60 μg/ml of culture medium. As seen in FIG. 6, none of the concentrations had any effect on the growth patterns of the two lymphoma lines. These results and co-immunoprecipitation studies of latexin-associated proteins discussed below, demonstrate that latexin acts to suppress lymphoma growth via a novel, non cononical mechanism distinct from its CPI activity. Potato CPI has been used previously to inhibit carboxypeptidase activity in mammalian cells and it has been shown to freely enter cells (Blanco-Aparicio). The doses of potato CPI chosen for the experiments were taken directly from a study in which the inhibitor was shown to inhibit the growth of pancreatic adenocarcinoma cells by directly interfering with the epidermal growth factor signaling pathway. Maximal growth inhibition was achieved at 30-50 μg/ml (x). Potato CPI can also be administered systemically to mice and because of its high chemical stability and cell membrane permeability, can have long-lasting effects in vivo. In a separate study potato CPI was injected intraperitoneally in a regimen of multiple injections over two weeks in mice. The effects on the size of the hematopoietic stem cell population in the bone marrow of these animals was determined. Stem and progenitor cell numbers were not affected by CPI administration. Thus, the mechanisms by which latexin regulates both stem cell population size and lymphoma growth inhibition reside in a novel pathway.

Next, latexin was tagged with a dual module of calmodulin and streptavidin which may be used sequentially, in tandem, to achieve very high levels of latexin purification from heterogeneous protein mixtures (Tandem Affinity Purification, TAP). TAP-tagged latexin was over-expressed in the multipotent hematopoietic progenitor cell line, FDCP-1, using an Sf beta-based retroviral expression vector, also containing GFP. Infected (GFP+) cells were purified from large-scale cultures using flow sorting. A control experimental arm was carried out using the same manipulations with a control vector identical to the latexin vector except that it contained the TAP dual module and GFP, but no latexin. Cell lysates of the resulting cells were then obtained, and latexin and its associated proteins were purified using the TAP purification method. Purified proteins were then electrophoresed. Parallel gels were stained using SYPRO Ruby or were silver stained.

A silver stained gel showed the presence of approximately a dozen proteins in the TAP-tagged latexin lane. Lysates from control vector-infected cells show only a single weak band. The TAP-m(ouse)latexin lane from the SYPRO Ruby-stained gel was then sliced into four segments which collectively contained the majority of the electrophoretic bands. The proteins from each segment were then eluted, subjected to enzymatic digestion, and the peptides separated using liquid chromatography and entrained for analysis using mass sptectrometry (LC-MS) by the Proteomics Facility at this university. The MS results were submitted to MASCOT for a database sequence similarity search. A compilation of the proteins identified by homology with at least two peptides from the digest are shown in Table 4. Contaminating keratins are not listed.

TABLE 4
Potential transcription factors for binding to the
latexin promoter CpG island

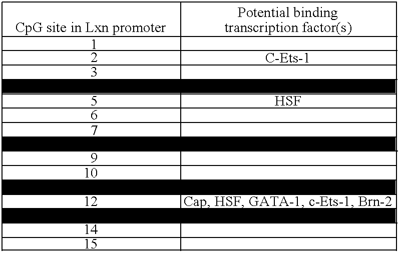

The TFSEARCH (www.cbrc.jp/research/db/TFSEARCH.html) program was used to predict putative transcription factors that may bind to the latexin promoter CpG island. Each transcription factor and its potential CpG binding site are listed. Several candidates co-localized with methylated CpG specific to the CD34+CD38− cells from the two AML patients (see FIG. 4b); these candidates (HSF-2, Ttk, V-myb, C-Rel and NF-κB) are highlighted and in bold font.

The presence of some of the protein targets in the list by means independent of LC-MS were confirmed, using, for example, co-immunoprecipitation and Western blotting. FIG. 9 shows Western blots of latexin bait and its ‘prey’ proteins following TAP purification from FDCP-1 lysates. The gels were reacted with antibodies directed against two candidate prey proteins listed in Table 4: ribosomal protein S3 (RPS3) and eEF1A1. Both show the presence of the cognate protein as a latexin-associated protein, thus validating the results from the LC-MS analysis. Conversely, antibodies to RPS3 and eEF1A1 were then used to immunoprecipitate their cognate proteins as bait. Western blots of the immunoprecipitated proteins were then probed for latexin as the prey. The presence of latexin using either RPS3 or eEF1A1 as bait confirms that latexin forms either a complex with each individually or that all three are members of a single complex (FIG. 10). Rabbit serum was used as a control, heterogeneous protein mixture in the immunoprecipitation reactions using anti-RPS3 and anti-eEF1A1. Carboxypeptidase A was not identified as a prey protein in TAP-tag purifications of labeled latexin (the bait) in cell lysates of FDCP-1 cells, nor was latexin identified as prey when CPA was used as bait. These results are consistent with latexin acting through a non-canonical regulatory mode in hematopoietic cells.

eEF1A1 and its isoform eEF1A2 are elongation factors during the translation of nascent proteins. An oncogene status has been ascribed to them in some carcinomas and they are associated with cell proliferation in hepatocarcinomas. This association implicates an interaction with latexin.

RPS3 has recently been shown to be an important non-Rel protein component of the NF-kB complexes consisting of p65 homodimers and p50-p65 heterodimers. More specifically, RPS3 appears to play a “specifier” role in determining, via direct interaction with chromatin, the groups of genes activated by NF-kB. NF-kB, of course, is an important arm of the signaling pathway in B cell differentiation, and for that matter, in PAR4 functioning. Latexin determines the availability of RPS3 by either sequestering it or altering it. The regulation of its availability, in turn, affects NF-kB/RPS3 complexes and thus may affect the activation of specific genes via NF-kB transcriptional control. This hypothesis serves as a unifying theme inter-relating the three projects in this proposal. Exploring the connection between latexin, RPS3 and NF-kB will be an area of focus in the third specific aim.

### Example 2

Adult lymphomas, largely confined to those over the age of 50, are characterized by greatly enhanced lymphocyte proliferation and incomplete functional development. Because it was shown that there are significant perturbations in both mouse and humans in latexin expression in lymphomas, the effects of proliferative activation and apoptosis on latexin expression in normal mouse spleen cells were examined. Latexin not only affects HSC proliferation (negatively), but also apoptosis (positively). Accordingly, primary spleen cells were stimulated in vitro with a number of manipulations of normal B cell proliferation and apoptosis. For example, anti-IgM may be used to stimulate both proliferation and apoptosis, LPS to stimulate proliferation, differentiation, 1 g secretion and little apoptosis, and the two together to greatly enhance proliferation with little or no apoptosis.

Pilot time-course studies showed that modulation of latexin expression mirrored the effects on proliferation. At 48 hour culture time-point proliferative responses were maximal. Effects of multiple treatments on cell proliferation (³H-thymidine uptake), apoptosis (Annexin V staining), and latexin expression at the protein level (Western blots) were determined. B6 spleen cells were enriched for B cells by removing adherent cells by adherence to plastic, RBCs by hypotonic lysis, and T cells by resetting. Resulting cells were cultured for 48 hours in RPMI containing 10% fetal bovine serum. Additions were (from left to right in upper panel): medium alone, anti-CD40, 3 Db CPG oligonucleotide (1 μM), 1 a non-CPG control oligonucleotide (1 μM), LPS (1 μg/ml), anti-IgM F(ab)₂ (50 μg/ml), and LPS+anti-IgM. The upper panel shows the absolute number of viable and apoptotic cells recovered. Proliferation, as measured by tritiated thymidine incorporation (“CPM”), clearly mirrors the absolute number of cells recovered except for anti-IgM group where proportionally the number of apoptotic cells was highest (19%). Apoptosis was also high in the anti-CD40 and 3 Db CPG groups; 14 and 15%, respectively. Cells cultured with anti-CD40 showed an expression level roughly equivalent to cells cultured with LPS.

Anti-IgM produced a strong induction of latexin expression that was partially abrogated by the presence of LPS. (This effect has been seen in multiple experiments and at different timepoints.) The IgM finding fits with the finding that high latexin expression in HSC is associated with higher levels of apoptosis; and the LPS effect is consistent with an anti-proliferative effect of latexin in HSC. Also, the 3 Db CPG oligonucleotide had the strongest inductive effect on latexin expression, showed modest proliferative effects and high levels of apoptosis. This oligonucleotide has been shown to act on early progenitors including Pro B cells (Kim, J. M. et al. (2005) Int Immunol 17, 1525-1531; Nagai, Y. et al. (2006) Immunity 24, 801-812), indicating that latexin expression may be highest in early B cell progenitor cells as was found in HSC (Hardy, R. R. et al. (2007 Immunity 26, 703-714).

### Example 3

Young 8- to 12-week old female C57BL/6 (B6) and 7-week old female BALB/c mice were purchased from the Jackson Laboratories (Bar Harbor, Me.). Mice were kept in the animal facilities of the University of Kentucky under pathogen-free conditions according to N1H-mandated guidelines for animal welfare. They were provided acidified water and food ad libitum.

### Leukemia Cell Lines

Nine human leukemic cell lines (K562, Molt4, CCRF-CEM, J45.01, Jurkat, U937, HL-60, KG-1, and SupB15) and two mouse lymphoma cell lines (WEHI-231 and A20) were included in the study. The leukemia cell lines were maintained in IMDM supplemented with either 10% (K562) or 20% (KG-1, HL-60, and Sup-B15) fetal bovine serum (FBS), or RPMI medium with 10% FBS, 10 mM Hepes (Molt4, CCRF-CEM, J45.01, Jurkat, and U937), 0.05 mM β-mercaptoethanol, 80 U/mL penicillin, and 80 mg/ml streptomycin. The cells were incubated in a humidified atmosphere of 5% CO₂ in air at 37° C.

### Isolation of CD34+ and CD34+CD38− Cells

Primary AML cells were obtained from the peripheral blood of patients at the Markey Cancer Center. Normal bone marrow was obtained as discarded material following pathologic analysis, surgical marrow harvest, or from the National Disease Research Interchange (NDRI). CB was obtained from patients at the University of Kentucky Obstetrics Department or from the NDRI. All tissues were obtained with the approval of the respective institutional review boards and appropriate informed consent.

Marrow cells were depleted of erythrocytes by suspending in 150 mM NH₄Cl plus 10 mM NaHCO₃ for 5 minutes, followed by 2 washes with phosphate-buffered saline (PBS). Blood cells were subjected to Ficoll-Paque (Pharmacia Biotech, Piscataway, N.J.) density gradient separation to isolate the mononuclear white blood cell compartment. Resulting leukocytes from marrow or blood were then used for immunoaffinity selection and flow cytometric sorting. For CD34+ cell selection, the Miltenyi immunoaffinity device (VarioMACS) was used according to the manufacturer's instructions (Miltenyi Biotech, Auburn, Calif.). For CD34+CD38− cell selection, the cells were further stained with anti-CD34 and —CD38 antibodies (Pharmingen, San Diego, Calif.), and sorted using a triple-laser FACSVantage flow cytometer (Becton Dickinson Immunocytometry Systems, San Jose, Calif.). In some cases, leukocytes were cryopreserved at a concentration of 5×10⁷ cells/mL in freezing medium consisting of Iscoves modified Dulbecco medium (IMDM), 40% fetal bovine serum (FBS), and 10% dimethyl sulfoxide (DMSO).

### Quantitative Real-Time PCR

To measure the expression of latexin in leukemic cells, quantitative real-time PCR was performed. Identical numbers (200,000) of cells were used for total RNA extraction using RNeasy Mini kit (QIAGEN, Valencia, Calif.) according to the manufacture's instructions. Isolated total RNA was reverse transcribed into cDNA using random hexamers in TaqMan® reverse transcription solution (PN N8080234), and stored at −20° C. In real-time PCR reactions, primer and probe mix for human latexin were purchased from Applied Biosystems (Foster city, CA, USA). TaqMan® human glyceraldehyde-3-phosphate dehydrogenase (GAPDH) was served as an endogenous control to normalize latexin expression. PCR reactions were set up as per manufacturer's instructions using TaqMan® universal PCR master mix (PN 4304437). Analyses of gene expression were performed in single reporter assays in an ABI PRISM 7700 sequence detection system (PE Biosystems, Foster city, CA, USA).

### Western Blots

Cell samples were lysed at a concentration of 2×10⁷ cells/ml in a protein lysis buffer containing 10 mM Tris pH7.5, 50 mM NaCl, 30 mM sodium pyrophosphate, 50 mM NaF, 5 μM ZnCl₂, 1% Triton X-100, 2.8 [symbol] g/ml aprotinin (Sigma-Aldrich; St. Louis, Mo.), 1 mM phenylmethylsulfonyl fluoride (Sigma), 1 mM sodium vanadate (Na₃VO₄), 1 [symbol] g/ml pepstatin, and 1 μg/ml leupeptin (Oncogene Research, MA, USA). The lysate was incubated on ice for 30 minutes, and then centrifuged at 15,000×g for 10 minutes to remove debris. The resulting supernatant was aliquoted and stored at −80° C.

For western blots, protein lysates were thawed and mixed with running buffer and a reducing agent (Novex, San Diego, Calif., USA, per manufacturer's instructions), and heated at 95° C. for 5 minutes. Using the equivalent of 4×10⁵ cells per lane, samples were separated by denaturing PAGE (Novex, 10% bis-Tris gel), and electro-transferred onto immunobilon-P membranes (Millipore, Bedford, Mass., USA), which were subsequently blocked and probed with polyclonal rabbit anti-latexin Ig-G antibody at a 1:3000 dilution. This antibody was generated from the latexin-specific amino acid sequence CKHNSRLPKEGQAE at the carboxyl terminus, and was produced by Bethyl Laboratories, Inc (Montgomery, Tex.). The antibody for detection of human latexin was purchased from Abeam Inc. (Cambridge, Mass.), and used at a 1:2000 dilution. Primary antibodies were detected using alkaline phosphatase-conjugated secondary antibodies (Santa Cruz Biotechnology) and electro-chemifluorescent (ECF) reagent (Pharmacia Biotech) according to the manufacturer's instructions. Blots were visualized using a Molecular Dynamics STORM 860 system and Imagequant Software. Following the detection and quantification of latexin, the immunobilon-P membrane was sequentially stripped using 40% methanol and a buffer containing 100 mM β-mercaptoethanol, 2% sodium dodecyl sulfate, and 62.4 Tris-HCl to remove the ECF reaction product and antibodies, respectively. The stripped membrane was re-probed with anti-actin antibody (Sigma) at a 1:500,000 dilution, and detected as described previously.

### Genomic Bisulfite Sequencing

To investigate the methylation pattern of latexin promoter, CpG island analysis in the upstream sequence of latexin open reading frame was performed. The nucleotide sequence of latexin in the upstream region (−1000 bp) and the first 3 exons (+373 bp) were obtained from the Ensembl database (www.ensembl.org) with ID number ENSG00000079257. A CpG island search using the CpG island searcher website (http://www.uscnorris.com/cpgislands2/cpg.aspx) showed a 252 by segment (−208 by to +44 bp) in the upstream region of latexin sequence enriched for CpG repeats. The criteria for the 5 CpG island is: GC content>50%, a ratio of CpG to GpC>0.6, and a 200 by minimum length. Genomic DNAs were isolated using AquaPure Genomic DNA kit (Bio-Rad, Hercules, Calif.), and modified by sodium bisulfite using EpiTect® Bisulfite kit (QIAGEN, Valencia, Calif.). For the latexin promoter methylation study, primers were designed that could amplify a 423 by fragment in the upstream region of latexin containing CpG island. The forward primer sequence is 5′ GTTGGTGTTTGATAAGTATGTGG 3, and the reverse primer sequence is 5′ TTTAACCTTCTACACCTCAAACAC 3′. The annealing temperature for the primers was 52° C. for 2 minutes. Hot-start PCR, with a total cycle number of 30, was used in all PCR amplifications. Denaturation and extension cycles were maintained for 95° C., 30 seconds and 72° C., 1 minute, respectively. The amplified fragments were cloned into the pCR2.1-TOPO vector using TOPO TA Cloning Kit (Invitrogen Carlsbad, Calif.) and sequenced (MWG Technology) n≧3 clones for cell line and n≧8 clones for primary cells).

5-aza-2′-Deoxycytidine Treatment

To examine the correlation of promoter hypermethylation and latexin gene expression, leukemia cell lines shown to have a lack of or decrease in latexin expression were subjected to 5-aza-2′-deoxycytidine treatment. Cells were plated with 2 [symbol] M 5-aza-2′-deoxycytidine (Sigma-Aldrich; St. Louis, Mo.), and incubated for 4 days. The medium and the drug were replaced every 24 hours, and cells were harvested for RNA and DNA extraction 4 days after treatment.

Infection of WEHI231 and A20 Cells with a Lxn Expression Vector

Cloning of the mouse Lxn gene into the Sfbeta 91 retroviral vector and production of viral supernatant were performed. WEHI-231 and A20 cells, at a density of 1×10⁶ cells per 10 cm plate, were infected using 10 ml viral supernatant and 4 μg/ml of polybrene for 48 hours. The infected cells (GFP+ cells) were sorted and expanded in culture medium. The expanded GFP+population, if not used immediately, was cryopreserved at a concentration of 1×10⁷ cells/mL in freezing medium consisting of 80% fetal bovine serum and 20% dimethyl sulfoxide (DMSO).

### Growth Measurements of Retrovirally-Transduced Tumor Cells

Sorted GFP+A20 cells over-expressing either Lxn or Sfbeta 91 empty vector were counted on a hemacytometer using trypan blue dye exclusion, and 500,000 cells were seeded onto 25 cm² tissue culture flask in 4 mls of media. Cells were incubated in a humidified atmosphere of 5% CO₂ in air at 37° C., and subsequently counted on Day 3, 8, 12, 16, and 20. At each time point cells were split, and maintained at a concentration of 500,000 cells per 4 ml media.

The cumulative cell number was calculated from the cell counts, and the dilutions were made at each culture split. FACS analysis was also performed at each time-point to measure the percentage of GFP+ cells. For the in vivo measurement of tumor cell growth, various numbers (5,000; 25,000; 50,000, and 100,000) of GFP+ A20 cells over-expressing Lxn or Sfbeta 91 empty vector were injected in a 50 μl bolus subcutaneously in the shaved flank of BALB/cJ mice given 3.0 Gy of gamma radiation 4 hrs prior. Lymphomas were detectable by palpation 10-12 days post-injection, and all three dimensions of the tumors were measured with calipers on days 12, 14, 16, 19, and 21. The same individual made the measurements from day-to-day without knowing the treatment regimen the mice received. At day 21, host mice were euthanized, the lymphomas were excised, and single cell suspensions were made of each to determine the fraction of tumor cells expressing GFP.

### Cell Cycle and Apoptotic Analysis

The culture of GFP+A20 cells was maintained as described above. At each time-point, cell cycle analysis was measured by BrdU labeling using BrdU Flow Kit (Pharmingen, San Diego, Calif.) as per manufacturer's instruction. 10 [symbol] l of BrdU solution (1 mM) was added to 1×10⁶ cells in 1 ml culture medium, and incubated for 1 hour. The cells were fixed and permeabilized by Cytofix/Cytoperm Buffer, then treated with 30 [symbol] g DNase for 1 hour at 37° C. After washing with Perm/Wash buffer, cells were stained with PE-conjugated anti-BrdU antibody for 20 minutes at room temperature, washed, and supplemented with 20 [symbol] l of 7-AAD. The cells were analyzed by flow cytometry using a Facscan (Becton Dickinson Immunocytometry Systems, San Jose, Calif.).

Immunohistochemistry of A20 Cells Treated with Potato Carboxypeptidase Inhibitor (PCPI)

PCI was purchased from Sigma-Aldrich Co. (St. Louis, Mo.). FITC labeling of PCI was performed using FLUOROTAG™ FITC CONJUGATION KIT (Sigma-Aldrich) according to the manufacturer's instructions, and was used to treat A20 cells for fluorescence internalization assays. Cells were cultured on 22×22 mm microscope cover glasses (Fisher Scientific Co., Pittsburgh, Pa.) in media as described above, fixed onto the cover glasses with 1:1 methanol:acetic acid, and washed 3 times with PBS. The fixed cells were incubated with FITC-conjugated PCI at a concentration of 30 [symbol] g/ml at 37° C. for 30 minutes, washed, and stained with phycoerythrin (PE)-conjugated B220 and DAPI. The cover glasses coated with the A20 cell monolayer were flipped immediately and sealed onto glass slide. Images were taken with a Zeiss Axiovert-200 microscope using a high-resolution Zeiss digital camera (Carl Zeiss Inc., Thornwood, N.Y.)

Culture of A20 Cells with Potato Carboxypeptidase Inhibitor

To determine the effects of PCPI on A20 cell growth, A20 cells were seeded at a density of 6×10⁵/well in 6-well plates, and cultured overnight before the addition of 0, 5, 15, 30 or 60 mg/ml PCI. The cells were fed every 2 days with fresh medium (as above) containing the respective concentration of PCI, and viable cells were counted on a hemacytometer using trypan blue dye exclusion. Cells were split according to cell population size to maintain a cell concentration of 2-5×10⁶/ml and cultures were maintained for 12 days. The cumulative cell number was calculated from the cell counts and the dilutions made at each culture split.

### Statistical Analysis

Data were analyzed by either student t-test assuming unequal variance with P<0.05 (two-tail), or a one-way ANOVA.

### Loss of Latexin Expression in Malignant Cells

It was determined, by quantitative real-time PCR, latexin mRNA abundance in tumor cell lines, bone marrow cells and peripheral blood CD34+ cells from lymphoma and leukemia patients, and from normal donors. The patients tested included those with acute myeloid leukemia (AML), T cell pro-lympho leukemia (Pctl), plasma cell leukemia (PCL), acute T cell lymphoma (ATLL), and acute lymphoid leukemia (ALL, preB phenotype). The normal CD34+ cell samples were derived from cord blood (CB) and young (31 and 35 years) and old (85 and 97 years) adults. Compared with normal primitive hematopoietic cells, latexin mRNA expression was completely absent in most of the leukemic lines tested, including K562, Molt4, CRF-CEM, J45.01, Jurkat, and U937. latexin mRNA was decreased by at least two-thirds in primary malignant CD34+ cells, and was significantly diminished in HL-60, KG-1, and SupB15 cell lines. Using western blotting, latexin protein expression was also assessed in these samples, and nearly identical results were obtained. Quantification of latexin in CD34+ cells of all human normal and leukemic samples compiled to date were plotted which showed a significant decrease of latexin expression in malignant cells (P=0.03).

### Aberrant Promoter Hypermethylation of Latexin in Hematopoietic Malignancy

To assess whether the loss or decrease of latexin expression in malignant cell lines resulted from promoter hypermethylation, the status of the 5′ CpG island of the latexin gene surrounding its transcriptional start site was determined by genomic bisulfite sequencing. The CpG island spans from within the canonical 5′ promoter (−208 nt) to the transcription start site (+1 nt), and extends through the entire first exon (+44 nt); in all, there are 15 CpG dinucleotides within the CpG island. Almost complete methylation was seen in the J45.01, U937, Jurkat, Molt4, and CCRF-CEM (>90%) lines, which commensurately showed an absence of latexin expression. Scattered methylated CpG sites were found in the K562, KG-1, and SupB15 lines, which were linked to the weak expression of latexin. Although HL-60 had very low latexin expression, it was found that none of the CpG sites were methylated. This is probably due to other epigenetic mechanisms, such as histone deacetylation.

### Reactivation of Latexin Expression After Demethylating Reagent Treatment

To test the hypothesis that latexin promoter hypermethylation in malignant cell lines might be involved in the loss of expression, the effect of 5-aza-2′-deoxycytidine, a demethylating reagent, on latexin expression was studied. After treatment with 2 μM 5-aza-2′-deoxycytidine for 4 days, latexin gene expression was reactivated in cell lines (K562, Molt4, CCRF-CEM, J45.01, Jurkat, and U937) that completely lacked latexin expression prior to treatment, and was significantly up-regulated in HL-60, KG-1, and SupB15 lines.

### Latexin Promoter Methylation in Primitive Normal and Leukemic Stem and Progenitor Cells

The latexin promoter methylation pattern in progenitor (CD34+) and stem cell-enriched populations (CD34+CD38−) from normal individuals and leukemia patients was determined. Bisulfite sequencing was conducted on 8-9 individual clones isolated from bone marrow CD34+ cells of two normal donors of disparate ages (31 and 85 years) and from peripheral blood CD34+CD38− cells from two AML patients in blast crisis. In contrast to the two normal subjects, several CG dinucleotides were methylated in cells of the two AML patients, including CpG dinucleotides at positions 4, 8, 10, 11, and 13.

### Potential Transcription Factors Specific to Methylated CpGs

To further study the transcriptional regulation of human latexin, the Transcription Factor Search Website, TFSEARCH (www.cbrc.jp/research/db/TFSEARCH.html) was used to predict putative transcription factor-binding sites upstream of the latexin translation start site, especially in the CpG island. Numerous potential transcription factor binding sites were identified within this region. Several candidates, including HSF-2, Ttk, V-myb, C-Rel, and NF-κB, are particularly interesting because they co-localize with the methylated CpG specific to the CD34+CD38− cells from the two AML patients. Specifically, methylation of the 4^{th} CpG impacts the binding of HSF2, the 8^{th} CpG is involved in Ttk binding, and methylation of the 11^{th} through the 13^{th} CpG affects numerous transcription factors, including v-Myb, NF-κB, and c-Rel.

### Growth Suppression of Mouse Lymphoma Cell Lines In Vitro and In Vivo Following Ectopic Lxn Expression

To distinguish between a correlative and causative relationship between Lxn expression and tumor development, it was considered whether or not the re-initiation of Lxn expression affected the growth rate of malignant cells in vitro and in vivo. To that end, Lxn was ectopically expressed, using a retroviral expression vector, in the mouse A20 lymphoma cell line, a BALB/c-derived IgG+ve B cell lymphoma that expresses MHC class I and II H-2d molecules, but lacks Lxn expression. As a control, the same vector backbone, which lacked the Lxn gene, was used. Both vectors contained green fluorescent protein (GFP), which is selectable by flow cytometric cell sorting and serves as a marker of infected cells. A20 cells were retrovirally infected in vitro, GFP positive cells were purified by sorting, and their growth patterns in vitro and in vivo were determined.

It was shown that cultures of A20 cells infected with the Lxn expression vector contained only about half the number of cells at day 3 relative to A20 cells infected with the control (empty) vector or uninfected control cells (A20 control). The growth suppression by Lxn over-expression was exponentially amplified during subsequent days of culture, and, by day 20, nearly 16-fold less tumor cells were present in the Lxn over-expressing group than in the control cultures (right panel). The fraction of GFP+ cells remained at 90-100% throughout the 20 days of culture in both the Lxn vector- or control vector-infected cells. A day 20 of culture neither uninfected A20 cells nor A20 cells infected with the control (empty) vector expressed detectable latexin protein, whereas in the western blot a strong latexin band was evident in lysate of cells infected with the Lxn vector (upper band).

100,000 GFP+A20 cells, uninfected or infected with either the Lxn expression vector or the GFP only control vector, were injected subcutaneously into the flanks of BALB/c mice. When tumors were first palpable (day 12), the Lxn-expressing cells caused significantly smaller tumors (filled circles; P<0.005), and, when the experiments were terminated (day 21), the tumors in the Lxn vector-injected group averaged only 40% of the volume of tumors in the two control groups (P<0.05). At day 21, the fraction of tumor cells expressing GFP was determined. Virtually all of the tumor cells in the Lxn and control vector groups were GFP+. More to the point, western blots confirmed strong expression of Lxn, as was observed in the cells analyzed after 20 days in culture. Thus, the reduction in tumor growth was due to durable Lxn expression in the tumor cells.

To explore the effects of cell dose on tumor size and latency prior to palpability, graded doses of control or Lxn-expressing A20 cells were injected. At inoculum sizes of 5,000 and 25,000, the Lxn-expressing cells not only displayed in more impressive suppression of tumor size than the 100,000 cell inoculum, but resulted in delayed onset of measurable-sized tumors. At day 21, the reduction in tumor size caused by ectopic latexin was 83% and 63% at the 5,000 and 25,000 cell doses, respectively. Host animals were necropsied for evidence of gross metastases to the spleen, thymus, and liver. No evident tumors were found in any of the treatment groups. Similarly, flow cytometry detected no GFP+ cells in these anatomical sites.

### Lxn Inhibits Tumor Cell Growth by Increasing Apoptosis, but not Via its Canonical Function

In studies of normal hematopoietic stem cell, high Lxn expression was associated with increased apoptosis and decreased proliferation. To determine if these mechanisms were involved in the results with the tumor cells, these two parameters were measured in latexin or control vector-infected A20 cells throughout 21 days of culture. Flow cytometric analysis of cells stained with BrdU and 7AAD distinguishes cell subsets that are apoptotic (A), necrotic (N), or residing in G0/G1, S and G2/M phase of cell cycle. Enumeration of each subset during the course of cell culture (FIG. 6b) indicates dramatically increased apoptosis in ectopic Lxn expressing cells, especially at the early phases of tumor growth. For example, 10-fold more Lxn-expressing tumor cells were undergoing apoptosis at day 5 than control cells (10.6% vs 1%). However, no significant difference in proliferation rate or in numbers of necrotic cells was observed between the Lxn over-expressing and control cells (data not show). These results point to apoptosis as the major mechanism in latexin-mediated tumor suppression.

The only presently known function of latexin is its role as the sole carboxypeptidase A (CPA) inhibitor in mammalian cells. Therefore, to test whether or not the suppressive effect of ectopic Lxn expression on A20 and WEHI231 cell growth was due to its canonical inhibitory activity, the following experiments were performed. Potato carboxypeptidase A inhibitor (PCPI), a 39 amino acid protein that strongly inhibits mammalian CPA, was added to cultures of A20 and WEHI231 cells at concentrations ranging from 5 to 60 mg/ml. None of the concentrations had any effect on the growth patterns of the tumor lines, despite the continuous presence of PCPI for 12 days of culture. To rule out the possibility that PCPI failed to inhibit tumor growth because it did not enter the cells, it was labeled with fluorescein isothyiocyanate (FITC). PCPI is plentiful in the cytosol, but is not found in the nucleus. The doses of PCPI chosen for the above experiments were taken directly from a study in which the inhibitor was shown to inhibit the growth of pancreatic adenocarcinoma cells by directly interfering with the epidermal growth factor signaling pathway(18). Maximal growth inhibition was achieved at 30-50 mg/ml. Thus, the mechanisms by which latexin regulates both stem cell population size and lymphoma growth inhibition reside in a novel pathway involving apoptosis.

### Example 4

Using inherent differences between mouse strains in lifespan and hematopoietic stem cell (HSC) parameters, a link has been proposed between stem cells, tumorigenesis and lifespan (Liang, Y. et al. (2008) Exp Cell Res; Van Zant, G. et al. (1990) J. Exp. Med. 171, 1547-1565; Waterstrat, A. et al. (2008) Mechanisms of stem cell aging. In Telomeres and Telomerase in Ageing, Disease, and Cancer (Rudolph, K. L., ed) pp. 111-140, Springer-Verlag, Berlin). A genetic approach designed to uncover the underlying stem cell regulatory genes has been proposed. (Geiger, H. et al. (2001) Blood 98, 2966-2972; Liang, Y. et al. (2003) Curr Opin Hematol 10, 195-202; Van Zant, G. et al. (2003) Exp Hematol 31, 659-672).

Embryo-aggregated chimeras were studied between the commonly used DBA/2 (D2) and C57BL/6 (B6) mouse strains. The former are short-lived (540 da median lifespan for females) and have comparatively more HSCs in their bone marrow as young adults. In contrast, females of the B6 strain live 51% longer but have a HSC population, as young adults, that is one-third to one-seventh the size of that of D2 animals, depending on the quantitative assay used. During their lifespans, the relative size of the HSC populations is reversed: HSC numbers increase steadily throughout the lifespan of B6 mice, whereas in D2 they reach a peak at about 1 year and then steadily decline (de Haan, G. et al. (1999) Blood 93, 3294-3301). Moreover, as young adults, HSCs in D2 mice replicate more frequently than do those in young B6 marrow. When HSCs of the 2 strains were studied in the context of the cellular chimeras, it was found that despite their existence in a common environment and exposure to the same extrinsic signals, they behaved quite differently during aging. After about a year of stable contributions to lympho-hematopoiesis, D2 HSCs faltered and B6 HSCs began to play a larger role in hematopoiesis. By 3 years of age all blood cell production was the result of B6 HSC differentiation. D2 contributed no mature blood cells of any type. This showed that lifespan and the parameters considered to be important are HSC numbers at different ages and the differential rates of HSC proliferation (Van Zant, G. et al. (1990) J. Exp. Med. 171, 1547-1565).

The experiments in chimeras also led to the observation that several important properties of HSCs were cell-intrinsic and demonstrated that a comparative genetic analysis might get at the underlying genes responsible for the strain-specific phenotypes. A genetic mapping panel of BXD recombinant inbred strains was generated from B6 and D2 progenitor strains to carry out a linkage analysis between genotype and several of the more obvious HSC phenotypic differences between the progenitor strains.

Three major QTL contributed to the phenotypic variation between these two strains in the size of the HSC population in young adults, one of which was on Chromosome 3. Reciprocal Chr 3 congenic mice showed that introgressed D2 alleles increased HSC numbers due to enhanced proliferation and self-renewal, and reduced apoptosis, whereas B6 alleles had opposite effects.

Using oligonucleotide arrays, real-time PCR and Western blots, Lxn was identified as a gene whose differential expression at transcript and protein levels was associated with the allelic polymorphisms (Liang, Y. et al. (2007) Nat Genet. 39, 178-188). Expression levels were inversely correlated with HSC numbers, thus latexin is a negative regulator and ectopic expression of Lxn using a retroviral vector decreased stem cell population size. Clusters of single nucleotide polymorphisms (SNPs) were identified upstream of the Lxn transcriptional start site, at least two of which are associated with potential binding sites for transcription factors regulating stem cells, including NF-kB. Thus, promoter polymorphisms between the B6 and D2 alleles may affect Lxn gene expression and consequently influence the population size of hematopoietic stem cells. It was shown that latexin regulates stem cell population size in the bone marrow via proliferation, self-renewal and apoptosis. It was further shown in mouse strains congenic for the B6 and D2 alleles of latexin that the B6 allele, hypermorphic with respect to latexin expression, is associated with low proliferation within the HSC compartment (Lin-Sca-1+ kit+ and cobblestone area forming cells at day 35, respectively). The D2 allele (hypomorphic for latexin expression) on a B6 background imparts higher self-renewal in 4 serial transplantations and is associated with greater survival in the quaternary recipients. D2 alleles were associated with low apoptosis, and B6 alleles were associated with a high apoptotic rate in the B6 background strain.

All references cited above are incorporated herein in their entirety for all purposes.

What is claimed is:

1. A method of treating cancer comprising administering a pharmaceutical composition comprising an agent which increases the expression and/or the activity of latexin,
wherein latexin includes a latexin polynucleotide variant and/or a latexin polypeptide variant that interacts with the agent or substitutes and derivatives thereof.

2. The method of claim 1, wherein the latexin polynucleotide variant has at least 70% sequence similarity to SEQ ID NO: 1.

3. The method of claim 1, wherein the latexin polypeptide variant has at least 70% sequence similarity to SEQ ID NO: 2.

4. The method of claim 2, wherein the cancer is hematopoietic.

5. The method of claim 4, wherein the hematopeotic cancer is lymphoma or leukemia.

6. A method of inhibiting tumor growth and/or metastases comprising comprising administering an agent which increases the expression and/or the activity of latexin to a subject in need thereof in a therapeutically effective amount sufficient to inhibit tumor growth and/or metastases,
wherein latexin includes a latexin polynucleotide variant and/or a latexin polypeptide variant that interacts with the agent.

7. A method of promoting apoptosis in tumor cells comprising administering an agent which increases the expression and/or the activity of latexin to a subject in need thereof in a therapeutically effective amount to promote apoptosis in tumor cells,
wherein latexin includes a latexin polynucleotide variant and/or a latexin polypeptide variant that interacts with the agent.

8. The method of claim 5, wherein the leukemia is chronic myelogenous leukemia, acute myelogenous leukemia, adult acute lymphoblastic leukemia, mature B-cell acute lymphoblastic leukemia, chronic lymphocytic leukemia, prolymphocytic leukemia, or hairy cell leukemia.

9. The method of claim 5, wherein the tumor is a melanoma and the subject is further subjected to surgery, isolated limb perfusion, regional chemotherapy infusion, systemic chemotherapy, or immunotherapy with a second antibody or antisera to treat the melanoma.

10. The method of claim 6, wherein the metastases is a metastasis to brain, lung, liver, or bone.

11. The method of claim 6, wherein the metastasis is to lung, and the tumor is a melanoma.

12. A combination therapy for inhibiting tumor growth and/or metastatic progression and/or development of metastases comprising administering an agent which increases the expression and/or the activity of latexin, wherein latexin includes a latexin polynucleotide variant and/or a latexin polypeptide variant that interacts with the agent; and
a chemotherapeutic, an immunotherapeutic, and/or radiation therapy.


SEQUENCE LISTING (5 sequences)

SEQ ID NO: 1 - DNA, 1113 bp, Homo sapiens
agaagcagtcagcccagggctctcggatgcagggagcctgggcccaaacagcagcttccggagtcggaaggagctgaggaagaagacagactgaaggagcttgcgacttttccgcctcggcaaccggacccagcagcaagcaggacgggcggcgctctgctactggtcccgttaagccagagtagcccaagccctgaagtcactgctcatccggaatggaaatcccgccgaccaactacccagcctccagggcggccttggtggcacagaactacatcaactaccagcaggggaccccgcacagggtgtttgaggtgcagaaggtcaaacaagccagcatggaggatattccaggaagaggacataagtatcgccttaaatttgctgttgaagaaattatacaaaaacaagttaaggtgaactgcacagctgaagtactttacccttcaacgggacaagaaactgcaccagaagtcaacttcacatttgaaggagaaactggaaagaatccagatgaagaagacaacacattttatcaaagacttaagtccatgaaggaaccgctagaagcacaaaatattccagacaattttggaaatgtatctccagaaatgacgctcgttctacatttagcctgggttgcctgtggttatataatatggcaaaattctactgaagacacatggtataaaatggtaaaaattcaaactgtcaagcaagtgcaaagaaatgatgactttattgaattagactacaccattctacttcataatatagcatctcaggagattattccctggcaaatgcaagttctctggcatccacaatacggcactaaagtaaaacataatagccgtctgccaaaggaagtacaactggaataaacaaaaaccctaacactggaagtgtaaacatgtctattgatgtgtatgccaatttcactggcatctagcttatgaggccaaataatcccaaagtgtcactttatataaatgtcttgattacagtatagaactttatagagtccataatacaaagtatcactacataaaaatgtctttaaaacagtaatagtggtatgtatatccaaaataaaaagcttcaatttcagcc

SEQ ID NO: 2 - protein, 222 aa, Homo sapiens
MEIPPTNYPASRAALVAQNYINYQQGTPHRVFEVQKVKQASMEDIPGRGHKYRLKFAVEEIIQKQVKVNCTAEVLYPSTGQETAPEVNFTFEGETGKNPDEEDNTFYQRLKSMKEPLEAQNIPDNFGNVSPEMTLVLHLAWVACGYIIWQNSTEDTWYKMVKIQTVKQVQRNDDFIELDYTILLHNIASQEIIPWQMQVLWHPQYGTKVKHNSRLPKEVQLE

SEQ ID NO: 3 - protein, 14 aa, Artificial Sequence
  Description of Artificial Sequence Synthetic peptide
CKHNSRLPKEGQAE

SEQ ID NO: 4 - DNA, 23 bp, Artificial Sequence
  Description of Artificial Sequence Synthetic primer
gttggtgtttgataagtatgtgg

SEQ ID NO: 5 - DNA, 24 bp, Artificial Sequence
  Description of Artificial Sequence Synthetic primer
tttaaccttctacacctcaaacac